(19) United States
(12) Patent Application Publication
(10) Pub. No.: US 2010/0143336 A1
(43) Pub. Date: Jun. 10, 2010

(54) TOLL-LIKE RECEPTOR BINDING EPITOPE AND COMPOSITIONS FOR BINDING THERETO

(75) Inventors: Mark Heffernan, Dublin (IE); Christopher Locher, Boston, MA (US); William McCormack, Dublin (IE); Graham Donnelly, Dublin (IE)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: OPSONA THERAPEUTICS LIMITED, Dublin (IE)

(21) Appl. No.: 12/597,724

(22) PCT Filed: Apr. 28, 2008
(86) PCT No.: PCT/GB08/50309

§ 371 Date: Jan. 18, 2010

(30) Foreign Application Priority Data
Apr. 26, 2007 (IE) 2007/0310
Oct. 8, 2007 (IE) 2007/0720

(51) Int. Cl. A61K 39/395; C07K 14/00; C07K 16/00; G01N 33/53; A61K 39/00; A61P 37/04; A61P 9/10

(52) U.S. Cl. 424/130.1; 530/324; 530/387.1; 436/86; 424/185.1

(57) ABSTRACT

The present invention relates to the identification of an epitope defined by residues of Toll-like Receptor (2). Targeting a binding compound, such as an antibody to the epitope results in antagonism of Toll-like Receptor (2). Further provided by the invention is the use of polypeptide comprising amino acid residues which form the epitope for use in screening for binding compounds which bind thereto, as well as to polypeptide compound which comprise the amino acid sequences of the epitope for use as vaccine compositions, when the generation of antagonistic antibodies which have binding specificity to Toll-like Receptor (2) are required, for example in the treatment of Toll-like Receptor (2) mediated disease conditions.

### FIELD OF THE INVENTION

The present invention relates to the identification of a binding epitope present on Toll-like receptor 2 which, when bound by a ligand, can inhibit the function of Toll-like receptor 2 irrespective of whether a heterodimer is formed between Toll-like receptor 2 and Toll-like receptor 1 or between Toll-like receptor 2 and Toll-like receptor 6. The invention further extends to binding members such as antibodies and fusion proteins which have binding specificity to this epitopes.

### BACKGROUND TO THE INVENTION

Toll-like receptors (TLRs) form a family of pattern recognition receptors which have a key role in activating the innate immune response. 11 Toll-like receptors have been identified in humans to date. The members of the TLR family are highly conserved, with most mammalian species having between 10 to 15 TLRs.

Each TLR recognises specific pathogen-associated molecular signatures. Toll-like receptor 2 (TLR2, CD282, TLR-2) is activated by peptidoglycan, lipoproteins and lipoteichoic acid. Toll-like receptors are known to form either homodimers or heterodimers wherein each dimer has a different ligand specificity. TLR2 is involved in the formation of at least 2 different heterodimers. For example a heterodimer is formed with Toll-like receptor 1, this heterodimer being bound by ligands such as triacylated lipopetides. The heterodimer between TLR1 and TLR6 permits binding of ligands such as diacylated lipopeptides.

Ligand binding to TLR2 results in downstream signalling mediated by interaction with cytoplasmic adaptor proteins such as MyD88.

The immune response which results from TLR2 activation and signalling has implicated TLR2 as an important mediator in the development of many inflammatory and disease conditions. Accordingly, there is significant therapeutic interest in the modulation of the TLR2 signalling pathway. In particular, the recognition that chronic inflammation plays a key role in the development of conditions such as cardiovascular disease, as well as the identification that TLR2 mediated immune signaling has importance in inflammation and disease has resulted in a number of therapeutic approaches being designed which target TLR2 activation and signalling.

One such approach uses monoclonal antibodies, with binding specificity to the TLR2 receptor, to antagonise the function of TLR2. Although antibodies have been developed with binding specificity to TLR2, the present inventors have identified that in order to be effective in mediating global suppression of TLR2 mediated signalling, an antibody with specificity for TLR2 must be capable of binding to Toll-like receptor 2 in such a way as to inhibit signalling irrespective of whether a heterodimer is formed between TLR1 and TLR2 or between TLR2 and TLR6.

Following extensive experimentation, the inventors have identified a conformational and discontinuous binding epitope, which when bound by a binding member, serves to inhibit activation of the TLR2 receptor, irrespective of whether TLR2 has formed a heterodimer with TLR1 or TLR6. The epitope which is bound by the antagonistic binding member has been mapped and has been shown to comprise binding regions which are present at both the N-terminal and C-terminal portions of the Toll-like Receptor 2 molecule. The inventors have identified the utility of fragments of TLR2 which comprise the identified epitope in methods for the production of binding members which antagonise TLR2 activity. Such binding members may have particular utility in methods for inhibiting TLR2-mediated signaling and accordingly for the treatment and/or prevention of inflammatory and disease conditions.

### SUMMARY OF THE INVENTION

According to a first aspect of the present invention there is provided a fragment of the Toll-like Receptor 2 (TLR2, TLR-2, CD282) receptor which comprises at least one of the amino acid sequences comprising SEQ ID No:1, SEQ ID NO:2, SEQ ID NO:3, SEQ ID NO:4 and SEQ ID NO:5.

The TLR2 fragment of the invention essentially comprises less than the 784 amino acids of the amino acid sequence of human Toll-like Receptor 2 as defined herein as SEQ ID NO:53.

The invention further provides a polypeptide which is an epitope of Toll-like Receptor 2 and which comprises at least one of the amino acid sequences comprising SEQ ID No:1, SEQ ID NO:2, SEQ ID NO:3, SEQ ID NO:4 and SEQ ID NO:5.

The invention further extends to binding members which specifically bind to a Toll-like receptor 2 fragment of the invention. The binding member may be selected from the group comprising: a protein, a peptide, a peptidomimetic, a nucleic acid, a carbohydrate, a lipid and a small molecule compound.

In certain embodiments, the binding member is an antibody or an antibody binding fragment which specifically binds to at least one of the amino acid sequences comprising SEQ ID No:1, SEQ ID NO:2, SEQ ID NO:3, SEQ ID NO:4 and SEQ ID NO:5. In certain embodiments the TLR2 antagonist binds to a non-continuous epitope comprising amino acid residues derived from the amino and carboxyl terminals of the amino acid sequence of human TLR2. In certain embodiments the TLR2 antagonist binds to an epitope on TLR2 comprising amino acid residues 19 to 39 or 538 to 549 of SEQ ID NO:53.

In certain embodiments the antibody is selected from the group consisting of a human, humanised, chimeric, synthetic, camelid, shark or in-vitro antibody which has binding specificity to TLR2, or a binding fragment derived from any of the same. In certain embodiments the antibody is an antibody binding fragment selected from the group consisting of a Fab, scFv, Fv, or dAb fragment. In certain embodiments, the antibody molecule comprises two complete heavy chains and two complete light chains, or an antigen-binding fragment thereof.

In various further aspects, the invention provides a monoclonal antibody which has binding specificity for an epitope of TLR2, said epitope comprising at least one of the amino acid sequences comprising SEQ ID No:1, SEQ ID NO:2, SEQ ID NO:3, SEQ ID NO:4 and SEQ ID NO:5.

In certain embodiments, the antibody binds to both the C terminal domain and N terminal domain of TLR2 as defined herein. Furthermore, said antibody is not the anti-TLR2 antibody designated T2.5 or TL2.1.

In various further aspects, the present invention extends to the use of a peptide comprising at least one of the amino acid sequences comprising SEQ ID No:1, SEQ ID NO:2, SEQ ID NO:3, SEQ ID NO:4 and SEQ ID NO:5 in a method for generating a binding member which specifically binds to Toll-like Receptor 2 for use in the treatment of a TLR2-mediate inflammatory condition or disease.

The invention further extends to a method for the treatment or prophylaxis of a disease which is mediated by Toll-like Receptor 2 activation and/or signalling, the method comprising the step of administering to a subject in need of treatment a therapeutically effective amount of a binding member which specifically binds to an epitope comprising at least one of the amino acid sequences comprising SEQ ID No:1, SEQ ID NO:2, SEQ ID NO:3, SEQ ID NO:4 and SEQ ID NO:5.

In certain embodiments, the epitope comprises the amino acid sequence of SEQ ID NO:2 and SEQ ID NO:5.

In certain embodiments, the epitope comprises one of the amino acid sequence of SEQ ID NO:1 or 2 along with one of SEQ ID NO:3, 4 or 5.

Without wishing to be bound by theory, the inventors predict that the epitope which must be bound by a binding member in order to antagonise TLR2 activation and signalling comprises amino acid residues which are derived from both the C-terminal portion and N-terminal portion of the human Toll-like Receptor 2 polypeptide. Having predicted the three dimensional structure of human Toll-like Receptor 2, the inventors have surprisingly shown an epitope which comprises residues from the N-terminal and C-terminal portions of Toll-like Receptor 2. The inventors have identifies that this epitope is distinct from the Toll-like Receptor 2 ligand binding site. It is therefore predicted that the binding of a binding member to the epitope which has been mapped herein, results in a conformational change which prevents TLR2 ligand binding. This in turn prevents TLR2 activation and TLR2 mediated downstream signalling.

The inventors have further identified the utility of the epitope of the present invention in the provision of vaccine compositions which can be administered to subjects for use in the treatment of chronic inflammatory conditions and chronic diseases. Specifically, polypeptides comprising at least one of the amino acid sequences of the group comprising SEQ ID NO:1, SEQ ID NO:2, SEQ ID NO:3, SEQ ID NO:4 and SEQ ID NO:5 can be prepared and administered to a subject. The host immune response generated against these peptides will result in the production of antibodies which have binding specificity to the epitope of the invention. These antibodies will be characterised in that they will have binding specificity to the epitope of the invention. They will therefore function as antagonistic antibodies which have use in antagonising TLR2 function. Such antagonism has utility in methods for treating chronic inflammatory conditions or disease conditions which are mediated through Toll-like Receptor 2.

Accordingly a yet further aspect of the present invention provides a vaccine composition comprising a polypeptide which comprises at least one of the amino acid sequences selected from the group comprising SEQ ID NO:1, SEQ ID NO:2, SEQ ID NO:3, SEQ ID NO:4 and SEQ ID NO:5. The vaccine compositions of the invention do not extend to the administration of the complete human Toll-like Receptor 2 amino acid sequence as defined herein in SEQ ID NO:53.

As defined herein, the term “specifically binds”, “binds sepcificially” or “binding specificity” refers to the ability of a TLR2 modulator agent or TLR2 binding compound to bind to a target epitope present on TLR2 with a greater affinity than it binds to a non-target epitope. In certain embodiments specific binding refers to binding to a target epitope present on TLR2 with an affinity which is at least 10, 50, 100, 250, 500 or 1000 times greater than the affinity for a non-target epitope. In certain embodiments binding affinity is determined by an affinity ELISA assay. In certain embodiments affinity is determined by a BIAcore assay. In certain embodiments binding affinity is determined by a kinetic method. In certain embodiments affinity is determined by an equilibrium/solution method.

According to one embodiment TLR2 modulators, including TLR2 binding agents, such as TLR2 antagonists, bind to the epitope defined herein with high affinity, for example, with an affinity constant of at least about 10⁷ M⁻¹, typically about 10⁸ M⁻¹, and more typically, about 10⁹ M⁻¹ to 10¹⁰ M⁻¹ or stronger, and modulate, e.g., reduce and/or inhibit, one or more TLR2 biological activities in a TLR2 responsive cell and/or tissue.

A yet further aspect of the invention provides a binding epitope which, when specifically bound by binding member, such as an antibody, results in antagonism of Toll-like Receptor 2 activation and signalling function, said binding epitope comprising at least one of the amino acid sequences selected from the group comprising SEQ ID NO:1, SEQ ID NO:2, SEQ ID NO:3, SEQ ID NO:4 and SEQ ID NO:5.

Without wishing to be bound by theory, the present inventors predict that binding to this epitope by a binding compound such as an antibody causes the suppression and/or inhibition of the activation of Toll-like receptor 2 and/or of downstream signalling mediated by Toll-like receptor 2 irrespective of whether Toll-like receptor 2 forms a heterodimer with Toll-like receptor 1 or Toll-like receptor 6. Furthermore, the inventors predict that unlike previous suggestions in the prior art that the binding epitope comprises only a portion of the C-terminal domain of the extracellular domain of Toll-like Receptor 2, it is shown herein that the epitope which must be bound in order to mediate global suppression of TLR2 activity is a discontinuous conformational epitope which is comprised of amino acids derived from both of the N-terminal domain and the C-terminal domain of TLR2.

In certain embodiments, where the binding epitope comprises the amino acid sequences of SEQ ID NO:1 or 2 along with at least one of the amino acid sequence of SEQ ID NO:3 and/or SEQ ID NO:4 or SEQ ID NO:5 which comprises a continuous sequence of SEQ ID NO;3 and 4. The epitope of the invention can be defined as a conformational and discontinuous epitope. That is, the epitope is not solely derived from a linear sequence of amino acids.

The derived amino acid sequence of human Toll-like Receptor 2 is defined herein as SEQ ID NO:53 in FIG. 27. The full length human TLR2 sequence comprises 784 amino acids and is defined as Genbank Accession Number AAC 34133 (URL www.ncbi.nlm.nih.gov)). The murine form of Toll-like Receptor 2 is described herein as SEQ ID NO:54 as provided in FIG. 28. The sequence of murine TLR2 is available as Genbank Accession Number NP_{—}036035 (Mus musculus)).

In certain embodiments, the epitope may comprise the amino acid sequence shown in SEQ ID NO:1 which is present at residues 27 to 31 at the N-terminal region of the defined amino acid sequence of human Toll-like Receptor 2 (SEQ ID NO:53).

| | |
|---|---|
| SEQ ID NO: 1: | SLSCD |

In certain embodiments, the binding epitope comprises the amino acid sequence shown in SEQ ID NO:3 which is present at residues 19 to 39 at the N-terminal region of the defined amino acid sequence of human Toll-like Receptor 2 (SEQ ID NO:53).

| | |
|---|---|
| SEQ ID NO: 2: | KEESSNQASLSCDRNGICKGS |

In certain embodiments, the binding epitope comprises the amino acid sequence shown in SEQ ID NO:3 which is present at residues 538 to 549 at the C-terminal region of the amino acid sequence of at the N-terminal region of the defined amino acid sequence of human Toll-like Receptor 2 (SEQ ID NO:53).

| | |
|---|---|
| SEQ ID NO: 3: | CSCEFLSFTQEQQ |

In certain embodiments, the binding epitope comprises the amino acid sequence shown in SEQ ID NO:4 which is present at residues 550 to 563 at the C-terminal region of the amino acid sequence of the defined amino acid sequence of human Toll-like Receptor 2 (SEQ ID NO:53).

| | |
|---|---|
| SEQ ID NO: 4: | ALAKVLIDWPANYL |

In certain embodiments, the binding epitope comprises the amino acid sequence shown in SEQ ID NO:5 which is present at residues 538 to 563 at the C-terminal region of the amino acid sequence of the defined amino acid sequence of human Toll-like Receptor 2 (SEQ ID NO:53).

| | |
|---|---|
| SEQ ID NO: 5: | CSCEFLSFTQEQQALAKVLIDWPANYL |

The term “epitope” as used herein relates to a portion or portions of a macromolecule which is capable of being bound by a specific antibody, in this case, a portion of a polypeptide, in particular Toll-like receptor 2.

Epitopes generally consist of chemically active surface groups and have specific three dimensional structural characteristics, as well as specific charge characteristics. Typically, the TLR2 binding agent or binding compound antagonises the binding activity of TLR2 and as such binds to an epitope known as an inhibiting epitope or an inhibitory epitope. An “inhibiting” or “inhibitory” epitope means an epitope present on TLR2, that when bound by a binding compound such as a small molecule or an antibody, results in the loss of biological activity of TLR2.

Epitopes may be defined from contiguous or non-contiguous sequences of amino acid residues comprised within a polypeptide sequence. The term “contiguous epitope” defines an epitope comprised of a linear series of amino acid residues within a polypeptide which define the epitope. A “non-contiguous epitope”, which may also be referred to as a conformational and discontinuous epitope, is an epitope which is comprised of a series of amino acid residues which are non-linear in alignment, that is that the residues are spaced or grouped in a non-continuous manner along the length of a polypeptide sequence. A non-continuous epitope can be a discontinuous epitope wherein the amino acid residues are grouped into 2 linear sequences, or alternatively the non-continuous epitope can be a discontinuous scattered epitope wherein the residues which contribute to the epitope are provided in 3 or more groups of linear amino acid sequences arranged along the length of the polypeptide.

The TLR2 binding epitope defined herein has been identified as comprising amino acids which are present at both the N-terminal region of the TLR2 molecule and the C-terminal region of the mature extracellular domain of Toll-like receptor 2 (TLR2).

In certain further embodiments, the binding epitope is defined as a non-continuous epitope, said epitope being comprised of at least 10 amino acids wherein said amino acids are present in 2 or more linear sequences derived from SEQ ID NO:1, 2, 3, 4 or 5. In a further embodiment, the epitope is defined as a non-continuous epitope, said epitope being comprised of at least 20 amino acids wherein said amino acids are present in 2 or more linear sequences derived from SEQ ID NO:1, 2, 3, 4 or 5. In a further embodiment, the epitope is defined as a non-continuous epitope, said epitope being comprised of at least 50 amino acids wherein said amino acids are present in 2 or more linear sequences derived from SEQ ID NO:1, 2, 3, 4 or 5. In a further embodiment, the epitope is defined as a non-continuous epitope, said epitope being comprised of at least 40 amino acids wherein said amino acids are present in 2 or more linear sequences derived from SEQ ID NO:1, 2, 3, 4 or 5.

In still further certain embodiments of the present invention, the binding epitope comprises amino acid residues 27 to 31 of the amino acid sequence of SEQ ID NO:53 or of a sequence which has an amino acid identity of at least 85%, 90%, 95%, 96%, 97%, 98%, 99% or more thereto.

In still further certain embodiments of the present invention, the binding epitope comprises the amino acid residues of at least one of SEQ ID NO:1, 2, 3, 4 or 5 or a sequence which has an amino acid homology of at least 90%, 95%, 96%, 97%, 98%, 99% or more thereto.

In still further certain embodiments of the present invention, the binding epitope comprises amino acid residues 19 to 39 of the amino acid sequence of SEQ ID NO:53 or of a sequence which has an amino acid identity of at least 85%, 90%, 95%, 96%, 97%, 98%, 99% or more thereto.

In still further certain embodiments of the present invention, the binding epitope comprises amino acid residues 538 to 549 of the amino acid sequence of SEQ ID NO:53 or of a sequence which has an amino acid identity of at least 85%, 90%, 95%, 96%, 97%, 98%, 99% or more thereto.

In still further certain embodiments of the present invention, the binding epitope comprises amino acid residues 550 to 563 of the amino acid sequence of SEQ ID NO:53 or of a sequence which has an amino acid identity of at least 85%, 90%, 95%, 96%, 97%, 98%, 99% or more thereto.

In still further certain embodiments of the present invention, the binding epitope comprises amino acid residues 538 to 563 of the amino acid sequence of SEQ ID NO:53 or of a sequence which has an amino acid identity of at least 85%, 90%, 95%, 96%, 97%, 98%, 99% or more thereto.

The invention further extends to binding members which have binding specificity for a TLR2 binding epitope, said epitope comprising at least one of the amino acid sequences of SEQ ID NO:1, 2, 3, 4 or 5, wherein binding of said binding compound to the epitope results in antagonism of the function of Toll-like Receptor 2 activity.

Although the binding compounds of the invention are typically antibodies, or binding compounds derived from, or related to antibodies, in certain embodiments, the binding compounds may further comprise, but are not limited to at least one of the group comprising: proteins, peptides, peptidomimetics, nucleic acids, polynucleotides, polysaccharides, oligopeptides, carbohydrates, lipids, aptamers, small molecule compounds, and naturally occurring compounds.

In certain further embodiments, the invention provides antibodies, or similar binding compounds derived therefrom, which have high specific binding specificity for the binding epitope defined herein, said binding epitope comprising the amino acid sequences of SEQ ID NO:1, 2, 3, 4 or 5.

Typically the binding of said antibodies to the amino acid sequences defining the TLR2 binding epitope as defined herein results in a reduction, inhibition or antagonism of Toll-like receptor 2 activity, and in particular Toll-like receptor 2 activation and downstream mediated signaling. Typically, this reduction, inhibition or antagonism of Toll-like receptor 2 activity occurs regardless of whether Toll-like receptor 2 forms a heterodimer with Toll-like receptor 1 or Toll-like receptor 6.

By the term “Toll-like receptor 2 activation and downstream mediated signaling” it is meant any intracellular signaling pathway which is induced by activated TLR2. The signaling pathway may be a TLR-2 specific pathway, or may be a “shared” pathway, wherein the pathway may be activated by other sources, for example, pathways which contribute to the activation of the transcription factor NF-kappaB.

Such antibodies accordingly have utility in methods, uses and medicaments for the regulation or suppression of immune responses and in particular for the suppression of aberrant immune responses. Said antibodies have further utility in the regulation of immune cell associated disorders, for example autoimmune diseases. In further still embodiments, an anti-TLR2 antibody with binding specificity for the binding epitope of the present invention can be used as a targeting antibody to deliver a therapeutic or cytotoxic agent to a TLR2 expressing cell. In still further embodiments, an anti-TLR2 antibody with binding specificity for the binding epitope of the present invention can be used diagnostically. Accordingly, the antibodies provided in accordance with the present invention have utility in the diagnosis, treatment and prophylaxis of immune mediated conditions, examples of which are provided hereinafter.

Accordingly in a yet further aspect, the present invention provides an isolated antibody which has binding specificity to a binding epitope as defined according to the first aspect of the invention. Typically, the Toll-like receptor 2 is human Toll-like receptor 2. Alternatively, the Toll-like receptor 2 is murine Toll-like receptor 2. In further embodiments, the Toll-like receptor 2 is derived from any mammal other than a human or mouse, for example, a cow or rat. In certain further embodiments, the antibody of this aspect of the invention is cross-reactive, that is that it has binding specificity to Toll-like receptor 2 derived from different species.

The antibodies provided according to the present invention may have at least one of the following characteristics: (i) it is a monoclonal antibody or single specificity antibody, (ii) it is a human or in-vitro generated antibody, (iii) it binds to an epitope present on the C-terminal region of the extracellular domain of TLR2 and inhibits TLR2 mediated downstream signaling irrespective of whether a heterodimer is formed between TLR2 and TLR1 or TLR2 and TLR6, (iv) it binds to the epitope present on the extracellular domain of TLR2 with an affinity constant (Ka) of at least 10⁶M⁻¹.

In certain further embodiments, the antibody may have a dissociation constant (Kd) selected from the group consisting of: (i) a dissociation constant between 10⁻⁷M and 10⁻¹¹M, (ii) a dissociation constant of between 10⁻⁸M and 10⁻⁹M, (iii) a dissociation constant of between 10⁻⁹M and 10⁻¹⁰M, (iv) a dissociation constant of between 10⁻¹¹M and 10⁻¹²M.

The antibodies of the invention can be characterised in that they bind to a binding epitope of Toll-like receptor 2, said epitope comprising at least one sequence of amino acids selected from: SEQ ID NO:2, 3, 4, 5 or 6.

In certain further embodiments, the antibodies have binding specificity to a fragment of the extracellular domain of TLR2 as defined by residues 292 to 586 of the amino acid sequence of SEQ ID NO:53, wherein the fragment comprises at least 10, at least 20, at least 30, at least 40, at least 50, at least 100, or at least 200 amino acid residues contiguous to the amino acid sequence set forth in SEQ ID NO:53.

An antibody provided according to the present invention is typically a heterotetrameric antibody. Typically the antibody is a monoclonal antibody, however in certain embodiments, the antibody may be a polyclonal antibody mixture with specificity for the binding epitope of the present invention.

An antibody can comprise a dimer formed between a complex of a heavy and light chains. The antibody can comprise at least one complete heavy chain and one complete light chain or can assume an alternative structure, for example, the antibody can be an antibody fragment which comprises only an antibody binding fragment, such as a Fab, F(ab′)2, Fv or a single chain Fv (scFV). An antibody can be of an isotype selected from the group comprising; IgG, IgA, IgM, IgE. In particular, the antibody is of the isotype IgG and may be of the subclass IgG1, IgG2, IgG3 or IgG4.

In certain further embodiments, this aspect of the invention further provides for an isolated nucleic acid or vector which encodes the variable domains of the heavy and/or light chains of the immunoglobulin.

An antibody as described herein may be linked to another functional molecule such as a polypeptide, for example, a Fab fragment.

The antibodies provided by the present invention may be provided by a number of techniques. For example, a combinatorial screening technique such as a phage display-based biopanning assay may be used to in order to identify amino acid sequences which have binding specificity to the binding epitopes of the invention. Such phage display biopanning techniques involve the use of phage display libraries, which are utilised in methods which identify suitable epitope binding compounds in a procedure which mimics immune selection, through the display of antibody binding fragments on the surface of filamentous bacteria. Phage with specific binding activity are selected. The selected phage can thereafter be used in the production of chimeric, CDR-grafted, humanised or human antibodies.

In further embodiments, the antibody is a monoclonal antibody may be produced using any suitable method which produces antibody molecules by continuous cell lines in culture. Suitable methods will be well known to the person skilled in the art and include, for example, the method of Kohler and Milstein (Kohler et al. Nature, 256, 495-497. 1975), Chimeric antibodies or CDR-grafted antibodies are further provided within the scope of the present invention. In further embodiments, the antibodies of the invention may be produced by the expression of recombinant DNA in host cell.

In further embodiments, humanized antibodies are also provided. Humanized antibodies may be produced by the method of Winter as described in U.S. Pat. No. 5,585,089.

In further certain embodiments, the monoclonal antibodies may be human antibodies, produced using transgenic animals, for example, transgenic mice, which have been genetically modified to delete or suppress the expression of endogenous murine immunoglobulin genes, with loci encoding for human heavy and light chains being expressed in preference, this resulting in the production of fully human antibodies.

In order to induce an immune response which results in the production of human antibodies in a transgenic mouse which expresses human immunoglobulin genes, and antigen comprising the TLR2 epitope of the present invention may be administered to the mouse. In alternative embodiments, a synthetic equivalent of the epitope of the invention may be provided.

As such, in certain further embodiments, the epitope of the present invention is provided as a mimetic of the identified binding epitope which is defined by amino acid residues 292 to 586 of the amino acid sequence of SEQ ID NO:53. Such a mimetic epitope will generally comprise a three dimensional structure which is identical or similar to the epitope of the present invention. The mimetic epitope may have some or all of the amino acid residues which comprise the epitope of the invention replaced.

In a further aspect of the present invention, there is provided a method for the treatment and/or prophylaxis of an immune mediated condition, the method comprising the steps of:
providing a therapeutically effective amount of a Toll-like Receptor 2 binding compound which has binding specificity for a binding epitope comprising an amino acid sequence defined in SEQ ID NO:1, 2, 3, 4 or 5, and
administering the same to a subject in need of such treatment.

In certain embodiments the subject is a mammal, typically a human.

Typically, the immune-mediated condition is mediated in part or in totality by Toll-like receptor 2-mediated immune cell activation. In further certain embodiments, the immune mediated condition is a disease or condition in which signalling mediated by Toll-like receptor 2 mediates onset or progression of the disease condition.

In a further aspect there is provided a method for suppressing Toll-like receptor 2 functional activity, wherein said method comprises the step of:
providing a therapeutically effective amount of a binding compound which has binding specificity for a binding epitope comprising an amino acid sequence of SEQ ID NO:1, 2, 3, 4, or 5, and
administering the same to a subject in need of such suppression.

In certain embodiments the subject is a mammal, typically a human.

In certain further aspects, the antibodies provided by the invention can be used in methods for detecting the presence of Toll-like Receptor 2 in a sample, in-vitro. Typically, the sample is a biologically selected from the group comprising, but not limited to; serum, plasma, tissue and biopsy tissue. The antibodies of the invention have further application in methods for the in-vivo detection of the presence of TLR2, for example using imaging techniques which will be well known to the person skilled in the field. In such instances, the imaging technique may further involve the antibody being labelled, directly or indirectly, with a detectable substance to facilitate detection of the bound or unbound antibody.

A yet further aspect of the present invention provides a pharmaceutical composition comprising at least one binding member with a binding specificity for a binding epitope comprising an amino acid sequence as defined in SEQ ID NO:1, 2, 3, 4 or 5 along with at least one pharmaceutically acceptable carrier, diluent, solubiliser, emulsifier, preservative and/or adjuvant.

In certain embodiments, the pharmaceutical composition may further comprise a secondary therapeutic agent, such as, but not limited to a cytokine inhibitor, or an immunosuppressant.

In yet further aspects, the antibodies of the invention have utility in in-vivo and in-vitro methods for the delivery or targeting of a therapeutic agent to a TLR2 expressing cell.

In further aspects, the present invention extends to novel polypeptides which have affinity and binding specificity for the binding epitope of the present invention. Such polypeptide binding compounds therefore have utility in the regulation of the innate immune response. Novel TLR2 binding polypeptides may be identified by a number of techniques which will be well known to the person skilled in the art. For example, combinatorial screening, such as phage display bio-panning may be used to identify polypeptides which have binding specificity to the TLR2 binding epitope of the present invention.

In yet further aspects, the present invention provides assay methods for use in screening compounds for use in identifying compounds which exhibit binding affinity to the TLR2 epitope of the present invention. The interaction of such molecules with the binding epitope of the present invention may be useful in a therapeutic and prophylactic context.

It is well known that pharmaceutical research leading to the identification of a new drug may involve the screening of a very large number of candidate substances, both before and even after a lead compound has been found. Such means for screening for compounds which have binding affinity for the epitope of the present invention are further provided by the present invention. Compounds identified as binding compounds of the epitope of the present invention represent an advance in the therapy in these areas as they provide basis for design and investigation of therapeutics for in vivo use.

In various further aspects, the present invention relates to screening and assay methods and to compounds identified thereby, wherein said binding compounds have affinity and binding specificity for the epitope of the invention.

Thus, a further aspect of the present invention provides the use of the binding epitope of the invention, or a mimetic thereof (including a fragment or derivative thereof) in screening methods or assays for use in identifying and/or obtaining binding members, for example a peptide, small molecule, aptamer or a chemical compound, which has binding specificity to the binding epitope of the invention.

In certain embodiments, a method according to this aspect of the invention may comprise the steps of:
providing a peptide defining the epitope of the invention, and
bringing the epitope into contact with a candidate substance, wherein said contact may result in binding between the epitope and the substance.

Binding may be determined by any number of techniques, both qualitative and quantitative which will be well known to the person skilled in the art.

A substance identified as a binding compound of the epitope of the invention may be a peptide or may be non-peptide in nature, for example a peptidomimetic. Non-peptide “small molecules” are often preferred for many in-vivo pharmaceutical uses. Accordingly, a mimetic or mimic of the TLR2 epitope binding compound identified according to the assays of this aspect of the invention may be designed for pharmaceutical uses. The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been determined, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can also be used in this modelling process.

In a variant of this approach, the three-dimensional structure of the TLR2 binding epitope compound and its binding partner are modelled. This can be especially useful where the binding compound and/or binding partner change conformation on binding, allowing the model to take account of the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in-vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in-vivo or clinical testing.

A further aspect of the present invention therefore provides an assay for assessing binding activity between the TLR2 binding epitope of the invention and a putative binding molecule which comprises the steps of:
bringing at least one candidate binding compound into contact with a putative binding molecule or other test substance, and
determining interaction or binding between the at least one candidate binding compound and the binding molecule or test surface,
   wherein binding between the at least one candidate binding compound and the binding molecule is indicative of the utility of the at least one candidate binding compound.

In certain further embodiments, the candidate binding compound may be selected from the group comprising, but not limited to; a peptide, for example an antibody or antibody fragment, a chemical compound, and a peptidomimetic.

In yet further embodiments, the candidate binding compound may be natural or synthetic chemical compounds used in drug screening programmes. Extracts of plants which contain several characterised or uncharacterised components may also be used.

A candidate binding compound which has affinity and binding specificity with the binding epitope of the present invention may be isolated and/or purified, manufactured and/or used to modulate TLR2 functional activity.

The precise format of the candidate binding compound screening assays of this aspect of the invention may be varied by those skilled in the art using routine skill and knowledge.

In yet further aspects, the invention extends to the use of combinatorial library technology (Schultz, J S (1996) Biotechnol. Prog. 12:729-743) which provides an efficient way of testing a potentially vast number of different substances for ability their ability to bind to an epitope or to modulate the activity of a binding compound which binds to an epitope. Prior to or as well as being screened for modulation of activity, test substances may be screened for ability to interact with the polypeptide, e.g. in a yeast two-hybrid system (which requires that both the polypeptide and the test substance can be expressed in yeast from encoding nucleic acid). This may be used as a coarse screen prior to testing a substance for actual ability to modulate activity of the polypeptide.

The amount of test substance or compound which may be added to an assay of the invention will normally be determined by trail and error depending upon the type of compound used. Typically, from about 0.01 to 100 nM concentrations of putative inhibitor compound may be used, for example from 0.1 to 10 nM. Greater concentrations may be used when a peptide is the test substance.

The inventors have further identified that the epitope of the present invention has utility in relation to methods for inhibiting or suppressing signalling which is mediated through Toll-like receptor 2 by providing a compound which competes with Toll-like receptor 2 for Toll-like receptor 2-specific binding compounds. Specifically, a compound is provided which comprises at least one of the amino acid sequences, or a fragment thereof, which have been defined herein as contributing to the discontinuous, conformational epitope of the present invention.

Accordingly, in a further aspect of the invention there is provided a method for the treatment and/or prophylaxis of an inflammatory process mediated by Toll-like receptor 2, the method comprising;
administering to a subject in need of such treatment, a therapeutically effective amount of a composition comprising a polypeptide comprising an amino acid sequence defined in SEQ ID NO:2, 3 or 4, the epitope being characterised in that binding thereto by a binding compound causes the suppression and/or inhibition of the activation of Toll-like receptor 2 and/or of downstream signalling mediated by Toll-like receptor 2 irrespective of whether Toll-like receptor 2 forms a heterodimer with Toll-like receptor 1 or Toll-like receptor 6.

In certain embodiments the polypeptide is provided in a soluble protein, for example as a fusion protein, such as an Fc fusion protein. In certain embodiments, the fusion protein comprises the polypeptide defining the TLR2 epitope conjoined to an Fc receptor binding polypeptide derived from an immunoglobulin, typically a human immunoglobulin.

The polypeptide encoding the Fc receptor binding polypeptide may be obtained by recombinant methods or alternatively may be produced synthetically. In a further embodiment, the Fc receptor binding domain may be obtained following proteolytic digestion of immunoglobulin molecules, for example by papain digestion of immunoglobulins. The antigenic peptide may be coupled by any method to Fc by any method known in the art including chemical linkages. For example, the conjugation can involve the use of chemical crosslinking molecules, such as the use of heterobifunctional crosslinking agents, such as succinimidyl esters, for example, 3-(2-pyridyldithio)propionate or succinimidyl acetylthioacetate (Molecular Probes Inc. Handbook, Chapter 5, section 5.3).

The polypeptide defining the TLR2 binding epitope of the invention, or a fragment thereof, may be modified to create derivatives by forming covalent or aggregative conjugates with other chemical moieties, such as glycosyl groups, polyethylene glycol (PEG) groups, lipids, phosphate, acetyl groups and the like. Covalent derivatives of the polypeptides of the invention can be prepared by linking the chemical moieties to functional groups on the amino acid side chains or at the N-terminus or C-terminus of the antigenic polypeptide.

The inventors have further shown that the blocking or suppression of Toll-like receptor 2 through the use of an antibody having affinity and binding specificity to a binding epitope comprising the amino acid sequences of SEQ ID NO:, 2, 3, 4 or 5 can suppress signalling pathways mediated downstream of TLR2 which induce the production of the cytokines IL-1beta and MIP-1alpha. The inventors predict that the expression of these cytokines contributes to the onset and progression of cardiovascular diseases such as atherosclerosis. The inventors have therefore identified that compounds which block or suppress the ability of the TLR2 to mediate downstream signalling which is causative of IL-1 beta and MIP-1alpha expression have utility in the prevention and/or treatment of cardiovascular diseases such as atherosclerosis.

In a further aspect the invention provides a method for the treatment and/or prevention of a cardiovascular disease, the method comprising the steps of:
providing a binding compound which has binding specificity to a binding epitope comprising the amino acid sequence of SEQ ID NO:1, 2, 3, 4 or 5 which is present on Toll-like receptor 2 and which inhibits TLR2 mediated activation and signaling irrespective of whether Toll-like receptor 2 forms a heterodimer with Toll-like receptor 1 or Toll-like receptor 6, and
administering a therapeutically effective amount of said composition to a subject in need of such treatment.

In certain embodiments, the cardiovascular disease may be selected from the group consisting of, but not limited to: atherosclerosis, heart failure, myocarditis, myocardial dysfunction in sepsis, viral myocarditis and diabetes related angiopathy.

Without being bound by theory, the inventors predict that inhibiting TLR2 function serves to suppress the expression of a number of cytokines and chemokines which are associated with the development and recurrence of cardiovascular disease. In certain specific embodiments, the cytokine may be IL-1beta. In further embodiments, the chemokine which is suppressed is MIP-1alpha. Accordingly, in various further embodiments the present invention provides for method for the suppression of the expression or IL-16 and/or MIP-1α. The invention further extends to the use of compounds which suppress TLR2 mediated production of IL-1β and/or MIP-1α.

In still further aspects, the present invention provides for the use of an antibody which has binding specificity to a binding epitope defined by amino acid residues 292 to 586 of the amino acid sequence of SEQ ID NO:1 which is present on Toll-like receptor 2 and which inhibits TLR2 mediated activation and signaling irrespective of whether Toll-like receptor 2 forms a heterodimer with Toll-like receptor in methods for the treatment of diseases.

In certain embodiments the disease is cardiovascular disease. In further embodiments the disease is an autoimmune disease. In still further embodiments the disease is an immune-mediated disease.

Accordingly, a yet further aspect of the invention provides for the use of an antibody which has binding specificity to a TLR2 binding epitope defined by amino acid defined in SEQ ID NO:1, 2, 3, 4, or 5 and which inhibits TLR2 mediated activation and signaling irrespective of whether Toll-like receptor 2 forms a heterodimer with Toll-like receptor in the preparation of a medicament for the treatment of disease.

In certain embodiments the disease is cardiovascular disease. In further embodiments the disease is an autoimmune disease. In still further embodiments the disease is an immune-mediated disease.

In a further embodiment, the present invention provides an epitope which can be targeted in order to mediate agonist activity, for example the production of pro-inflammatory cytokine production.

In a further embodiment, binding to the identified epitope may result in the induction of regulatory T cell (Treg) activity. The activation of the Treg population of T cells results in the suppression of the immune response. As such, binding to the epitope of the present invention may result in the upregulation of Treg production.

### BRIEF DESCRIPTION OF THE FIGURES

FIG. 1(a) shows a schematic showing the arrangement of the 14 leucine rich repeat regions, and the positioning of a central spacer region between leucine rich repeat 7 (LRR7) (shown as section 8) and LRR8 (shown as section 9), wherein section 1 is the N-terminal region of the TLR2 polypeptide, and section 16 is the C-terminal region of the TLR2 peptide. FIG. 1(b) shows the predicted three dimensional structure of Toll-like Receptor 2, and in particular the likely positioning of the N-terminal and C-terminal ends of the polypeptide, these interacting to provide a discontinuous, conformational epitope which can be bound,

FIG. 2 shows a schematic representation of the putative TLR2 tertiary structure, with boxes A and B showing the predicted interaction of cysteine (C) residues by way of disulphide bridges present at the N-terminal region and C-terminal region respectively of TLR2. The defined amino acid sequence of the associated area of relevance of the N-terminal region (box C) and of the C-terminal region (box D) is also provided, this sequence information being aligned with the relevant homologous area of TLR3, this showing cysteine conservation in these region.

FIG. 3 (top graph) shows a that the anti-TLR2 monoclonal antibody OPN-301 is able to inhibit TLR2 mediated signaling in response to the tri-acylated lipopetide Pam3CSK4 in U937 cells (a human leukemic monocyte lymphoma cell line) stimulated with 100 ng/ml Pam₃ CSK₄. FIG. 3 (bottom graph) shows that the inhibition of TLR2 signaling is dose dependent.

FIG. 4(a) to (d) shows that the anti-TLR2 monoclonal antibody OPN-301 specifically inhibits TLR2 signalling. U937 cells were stimulated with the TLR1/TLR2 heterodimer ligand Pam3CSK4, with the TLR2/TLR6 heterodimer ligand FSL-1, with the TLR4 agonist LPS, or with LMW HA. TNF-alpha production was only seen following LPS and LMW HA stimulation, this suggesting that TLR2 specific stimulation was being inhibited by OPN-301.

FIG. 5(a) to (c) show that OPN-301 inhibits TNF-alpha production following stimulation of cells with Pam3CSK4 (FIG. 5A) and FSL-1 (FIG. 5B), but not following LMW HA stimulation (FIG. 5C),

FIG. 6 shows that OPN-301 antibody, inhibits TNF-alpha from both human (U937) and murine (mouse J774) cells lines,

FIG. 7 shows further experimentation showing OPN-301 inhibition in Pam3CSK4 stimulated murine J774 cells,

FIG. 8 shows inhibition of TLR2-mediated TNF-alpha expression following Pam3CSK4 stimulation of human PBMC from 2 subjects (S1 and S2), wherein FIG. 8(a) shows TNF-alpha expression after 6 hours in subject 1 following administration of OPN-301, FIG. 8(b) shows TNF-alpha expression in a sample from subject 2 following administration with OPN-301, FIG. 8(c) shows TNF-alpha expression in subject 1 after administration with isotype control antibody IgG1, and FIG. 8(d) shows TNF-alpha expression following administration of isotype control antibody to subject 2,

FIG. 9 shows inhibition of TLR2-mediated IL-6 expression following Pam3CSK4 stimulation of human PBMC from 2 subjects (S1 and S2), wherein FIG. 9(a) shows IL-6 expression after 6 hours in subject 1 following administration of OPN-301, FIG. 9(b) shows IL-6 expression in a sample from subject 2 following administration with OPN-301, FIG. 9(c) shows IL-6 expression in subject 1 after administration with isotype control antibody IgG1, and FIG. 9(d) shows IL-6 expression following administration of isotype control antibody to subject 2,

FIG. 10 shows inhibition of TLR2-mediated IL-1 beta expression following Pam3CSK4 stimulation of human PBMC from 2 subjects (S1 and S2), wherein FIG. 10(a) shows IL-1 beta expression after 6 hours in subject 1 following administration of OPN-301, FIG. 10(b) shows IL-1 beta expression in a sample from subject 2 following administration with OPN-301, FIG. 10(c) shows IL-1 beta expression in subject 1 after administration with isotype control antibody IgG1, and FIG. 10(d) shows IL-1 beta expression following administration of isotype control antibody to subject 2,

FIG. 11 shows inhibition of TLR2-mediated IL-8 expression following Pam3CSK4 stimulation of human PBMC from 2 subjects (S1 and S2), wherein FIG. 11(a) shows IL-8 expression after 6 hours in subject 1 following administration of OPN-301, FIG. 11(b) shows IL-8 expression in a sample from subject 2 following administration with OPN-301, FIG. 11(c) shows IL-8 expression in subject 1 after administration with isotype control antibody IgG1, and FIG. 11(d) shows IL-8 expression following administration of isotype control antibody to subject 2,

FIG. 12 shows inhibition of TLR2-mediated MIP-1alpha expression following Pam3CSK4 stimulation of human PBMC from 2 subjects (51 and S2), wherein FIG. 12(a) shows MIP-1alpha expression after 6 hours in subject 1 following administration of OPN-301, FIG. 12(b) shows MIP-1alpha expression in a sample from subject 2 following administration with OPN-301, FIG. 12(c) shows MIP-1alpha expression in subject 1 after administration with isotype control antibody IgG1, and FIG. 12(d) shows MIP-1alpha expression following administration of isotype control antibody to subject 2,

FIG. 13(a) shows that Murine TLR2Fc inhibits TNF-alpha production from TLR agonist stimulated cells, FIG. 13 showing a control experiment using the isotype control antibody IgG2a.

FIG. 14 shows FACS analysis of binding of OPN-301 to wild type and mutated forms of TLR2,

FIG. 15 shows median FITC results shown the data of FIG. 14,

FIG. 16 shows FACS analysis of binding of OPN-301 to wild type and mutated forms of TLR2,

FIG. 17 shows median FITC results shown the data of FIG. 16,

FIG. 18 shows the response to different dosages of expression plasmid DNA. Cells were transfected with 1 ng, 10 ng and 100 ng of pUNO vector expressing TLR wild type and mutant versions of human TLR2. FIG. 18 shows cells stimulated with Pam3CSK4 (20 ng/ml).

FIG. 19 shows the response to different dosages of expression plasmid DNA. Cells were transfected with 1 ng, 10 ng and 100 ng of pUNO vector expressing TLR wild type and mutant versions of human TLR2. FIG. 19 shows cells stimulated with HKLM (10⁷ cells/ml).

FIG. 20 shows OPN301 blocking activity. Cells were transfected with 50 ng of pUNO vector expressing wild type and mutant versions of hTLR2 as described in material and methods. OPN301 and isotype control were added 30 minutes before stimulation where indicated. FIG. 20 shows cells were stimulated with Pam3CSK4 (20 ng/ml).

FIG. 21 shows OPN301 blocking activity. Cells were transfected with 50 ng of pUNO vector expressing wild type and mutant versions of hTLR2 as described in material and methods. OPN301 and isotype control were added 30 minutes before stimulation where indicated. FIG. 20 shows cells stimulated with Pam3CSK4 (20 ng/ml).

FIG. 22(a) shows the binding affinity of 15-mer peptides designed based on the human TLR2 amino acid sequence of 1 ug/ml of OPN-301 monoclonal antibody.

FIG. 22(b) shows a series of linear peptide sequences derived from the peaks of FIG. 22(a) wherein the sequences are numbered SEQ ID NO:12 to SEQ ID NO:28,

FIG. 23 identifies the extracellular, transmembrance, intracelulluar and 14 leucine rich repeat domains of human Toll-like Receptor 2,

FIG. 24 shows a graph wherein peaks illustrate CLIPS peptides which have a strong binding affinity to OPN-301,

FIG. 25 shows a table of CLIPS peptide, identified as SEQ ID NO:29 to SEQ ID NO:48, which have a high binding affinity to OPN301,

FIG. 26 shown an alignment of the amino acid sequences of SEQ ID NO: 29, 30, 31, 32, 33, 34, 35, 37, 38, 39, 40, 41, 43, 46, 48, 49, 50, 51 and 55,

FIG. 27 shows an alignment of the amino acid sequences of the amino acid sequence of human TLR3 and human TLR2,

FIG. 28 shows a three dimensional structure of TLR2, wherein the predicted positioning of sequences which are involved with a binding epitope to which a TLR2 antagonistic antibody can bind are identified,

FIG. 29 shows the amino acid sequence of human Toll-like Receptor 2 (SEQ ID NO:53),

FIG. 30 shows the amino acid sequence of murine Toll-like Receptor 2 (SEQ ID NO:54).

### DETAILED DESCRIPTION OF THE INVENTION

The present invention relates to the identification of a discontinuous, conformational binding epitope comprising amino acid resides present at both the N-terminal and C-terminal regions of Toll-like Receptor 2 (CD282). The defined epitope, when bound by a binding compound exhibiting binding specificity thereto, results in antagonism of the function of Toll-like Receptor 2.

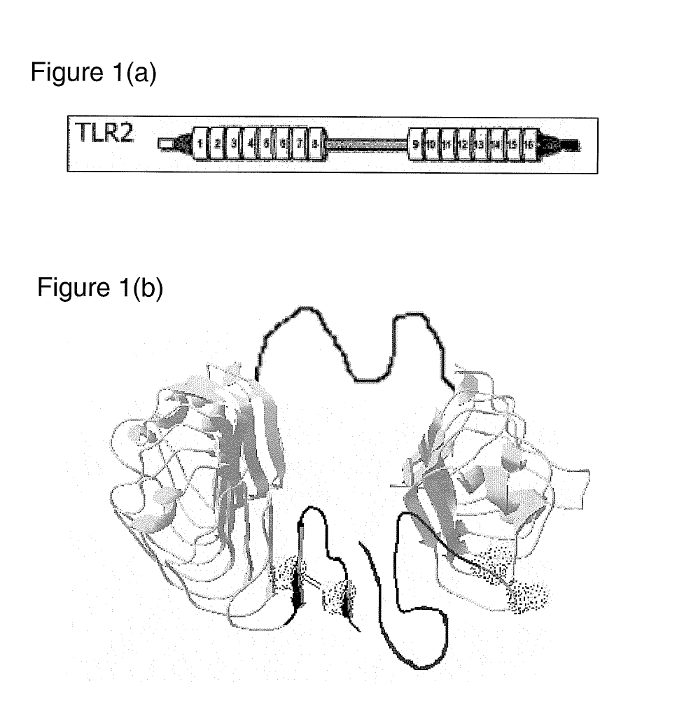

Without wishing to be bound by theory, the inventors have determined that the tertiary structure of the Toll-like Receptor 2 molecule comprises 14 leucine rich repeat regions (LRRs) and that the seventh and eight LRR is separated by a spacer region. A schematic of the regions and spacer predicted as forming the three dimensional structure of human TLR2 is shown in FIG. 1(a). FIG. 1(b) shows a predicted structure for human Toll-like Receptor 2 that is based on the structure of TLR3, whose crystal structure has been defined in the art.

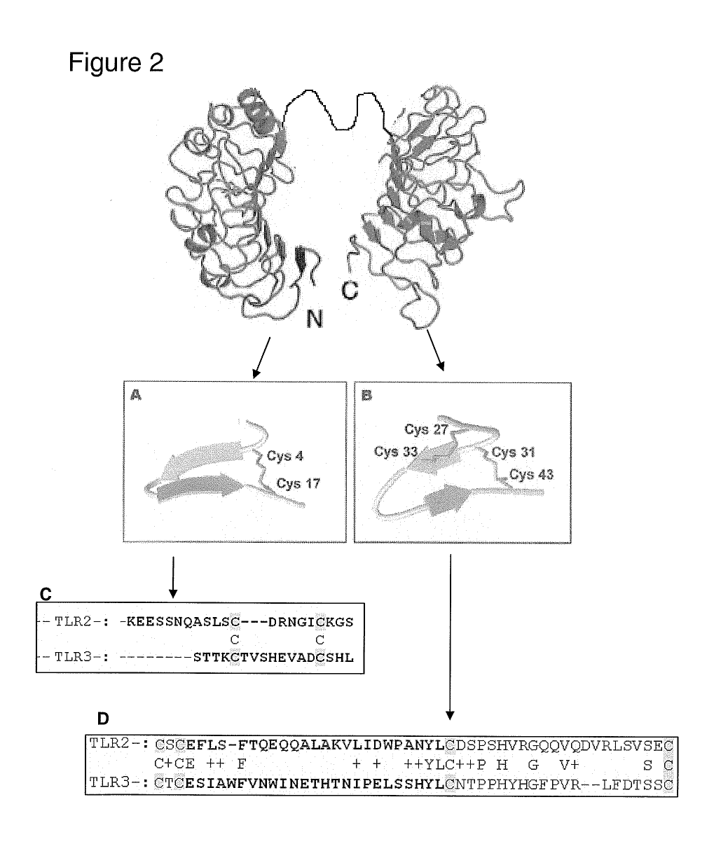

FIG. 2 also shows a further representation of the predicted crystal structure of Toll-like receptor 2. Again, without wishing to be bound by theory, this figure illustrates the general area of the N-terminal region and C-terminal region where the amino acids which are considered to contribute to the binding epitope are located (red, pink and blue). The identified binding regions have been shown to contain a number of cysteine (C) residues. FIG. 2 further shows how these cysteine residues may interact in relation to the tertiary structure of the TLR2 molecule. Further, the alignment shows that the cysteine residues in the N and C-terminal regions are also conserved in TLR3.

The determined binding epitope is therefore considered to comprise the amino acid residues provided in SEQ ID NO: 1 and/or 2 in conjunction with the amino acid sequence of SEQ ID NO:3 and 4, SEQ ID NO:5.

The invention further extends to the identification of binding compounds which have binding specificity for the identified epitope and further to methods of inhibiting TLR2 functional activity and to methods of treating TLR2-mediated disease and conditions using said compounds which exhibit a binding specificity to said epitope.

Compounds which have binding specificity to the binding epitope of the present invention have utility in the inhibition of number of immune mediated and disease conditions which are mediated or induced following signalling through Toll-like receptor 2.

As such, the invention provides compositions and methods for the treatment of; immune-mediated conditions, inflammatory conditions, pathogenic conditions and cancerous or malignant conditions.

In certain embodiments, the pathogenic condition is an infectious condition mediated by a bacteria. The bacteria may be a gram positive bacteria, or alternatively a gram negative bacteria. In a specific embodiment, the pathogenic disease is a sepsis-causing bacteria, and as such the compositions and methods have utility in the treatment of sepsis or septic shock. Such conditions may be referred to as endotoxin mediated conditions, for example sepsis, septic shock or septicaemia.

In certain embodiments, the inflammatory condition is a cardiovascular disease, which may be selected from the group consisting of, but not limited to: atherosclerosis, heart failure, cardiac inflammation, ischaemia, reperfusion, myocarditis, myocardial dysfunction in sepsis, stoke viral myocarditis, vascular injury, for example injury which may result from angioplasty, stenting and bypass grafting, and diabetes related angiopathy.

In certain embodiments, the inflammatory condition may include, but is not restricted to an autoimmune condition selected from the group consisting of, but not limited to; arthritis (including rheumatoid arthritis, juvenile rheumatoid arthritis, osteoarthritis, psoriatic arthritis), psoriasis, SLE, type I diabetes, type II diabetes, multiple sclerosis, allograft rejection, acute and chronic graft versus host disease, and tissue damage resulting from insult or injury.

Further conditions which may be treatable using the methods of this aspect of the present invention include; sepsis, including gram positive sepsis, cerebral malaria, gingivitis, diabetes mellitus, glomerular nephritis, myasthenia gravis, systemic lupus erythematosis, autoimmune thyroiditis, dermatitis (including atopic dermatitis and eczematous dermatitis), Sjogren's Syndrome, including keratoconjunctivitis sicca secondary to Sjogren's Syndrome, alopecia greata, allergic responses due to arthropod bite reactions, aphthous ulcer, iritis, conjunctivitis, keratoconjunctivitis, ulcerative colitis, asthma, allergic asthma, cutaneous lupus erythematosus, scieroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves opthalmopathy, sarcoidosis, primary biliary cirrhosis, uveitis posterior, interstitial lung fibrosis, Alzheimer's disease, coeliac disease, colitis, asthma and atopic disease.

### Antibodies

An “antibody” is an immunoglobulin, whether natural or partly or wholly synthetically produced. The term also covers any polypeptide, protein or peptide having a binding domain that is, or is homologous to, an antibody binding domain. These can be derived from natural sources, or they may be partly or wholly synthetically produced. Examples of antibodies are the immunoglobulin isotypes and their isotypic subclasses and fragments which comprise an antigen binding domain such as Fab, scFv, Fv, dAb, Fd, and a bi-specific antibody.

In further embodiments, the antibody may be a Camelid antibody, in particular a Camelid heavy chain antibody. Further the antibody fragment may be a domain antibody or a nanobody derived from a Camelid heavy chain antibody. In a further embodiment the antibody may be a shark antibody or a shark derived antibody.

As antibodies can be modified in a number of ways, the term “antibody” should be construed as covering any binding member or substance having a binding domain with the required specificity. The antibody of the invention may be a monoclonal antibody, or a fragment, derivative, functional equivalent or homologue thereof. The term includes any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in European Patent Application Publication Number EP 0,120,694 and European Patent Application Publication Number EP 0,125,023.

The constant region of the antibody may be of any suitable immunoglobulin subtype, however it is preferred that the antibody subtype is IgG1. However, in alternative embodiments, the subtype of the antibody may be of the class IgA, IgM, IgD and IgE where a human immunoglobulin molecule is used. Such an antibody may further belong to any subclass e.g. IgG1, IgG2a, 2b, IgG3 and IgG4.

Fragments of a whole antibody can perform the function of antigen binding. Examples of such binding fragments are; a Fab fragment comprising of the VL, VH, CL and CH1 antibody domains; an Fv fragment consisting of the VL and VH domains of a single antibody; a F(ab′)2 fragments, a bivalent fragment comprising two linked Fab fragments; a single chain Fv molecule (scFv), wherein a VH domain and a VL domain are linked by a peptide linker which allows the two domains to associate to form an antigen binding site; or a bi-specific antibody, which may be multivalent or multispecific fragments constructed by gene fusion.

A fragment of an antibody or of a polypeptide for use in the present invention, for example, a fragment of a TLR2 specific antibody, generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment of a TLR2 specific antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having TLR2 binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The term “antibody” includes antibodies which have been “humanised”. Methods for making humanised antibodies are known in the art. Methods are described, for example, in Winter, U.S. Pat. No. 5,225,539. A humanised antibody may be a modified antibody having the hypervariable region of a monoclonal antibody such as a TLR2 specific antibody and the constant region of a human antibody. Thus the binding member may comprise a human constant region.

The variable region other than the hypervariable region may also be derived from the variable region of a human antibody and/or may also be derived from a monoclonal antibody such as a TLR2 specific antibody. In such case, the entire variable region may be derived from murine monoclonal antibody a TLR2 specific antibody and the antibody is said to be chimerised. Methods for making chimerised antibodies are known in the art. Such methods include, for example, those described in U.S. patents by Boss (Celltech) and by Cabilly (Genentech). See U.S. Pat. Nos. 4,816,397 and 4,816,567, respectively.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184,187, GB Patent Number 2,188,638 or European Patent Number 0,239,400. A hybridoma or other cell producing an antibody may be subject to genetic mutation or other changes, which may or may not alter the binding specificity of antibodies produced.

### Production of Antibodies

Certain methodologies for producing antibodies which have an affinity and binding specificity for the TLR2 epitope of the present invention are described hereinbefore.

The antibodies or antibody fragments of and for use in the present invention may also be generated wholly or partly by chemical synthesis. The antibodies can be readily prepared according to well-established, standard liquid or, preferably, solid-phase peptide synthesis methods, general descriptions of which are broadly available and are well known by the person skilled in the art. Further, they may be prepared in solution, by the liquid phase method or by any combination of solid-phase, liquid phase and solution chemistry.

Another convenient way of producing antibodies or antibody fragments suitable for use in the present invention is to express nucleic acid encoding them, by use of nucleic acid in an expression system.

Nucleic acid for use in accordance with the present invention may comprise DNA or RNA and may be wholly or partially synthetic. In a preferred aspect, nucleic acid for use in the invention codes for antibodies or antibody fragments of the invention as defined above. The skilled person will be able to determine substitutions, deletions and/or additions to such nucleic acids which will still provide an antibody or antibody fragment of the present invention.

Nucleic acid sequences encoding antibodies or antibody fragments for use with the present invention can be readily prepared by the skilled person using the information and references contained herein and techniques known in the art (for example, see Sambrook et al. (1989), and Ausubel et al, (1992)), given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding antibody fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The nucleic acid may be comprised as constructs in the form of a plasmid, vector, transcription or expression cassette which comprises at least one nucleic acid as described above. The construct may be comprised within a recombinant host cell which comprises one or more constructs as above. Expression may conveniently be achieved by culturing under appropriate conditions recombinant host cells containing the nucleic acid. Following production by expression the antibody or antibody fragments may be isolated and/or purified using any suitable technique, then used as appropriate.

Systems for cloning and expression of a polypeptide in a variety of different host cells are well known. Suitable host cells include bacteria, mammalian cells, yeast, insect and baculovirus systems. Mammalian cell lines available in the art for expression of a heterologous polypeptide include Chinese hamster ovary (CHO) cells, HeLa cells, baby hamster kidney cells, NSO mouse myeloma cells. A common, preferred bacterial host is E. coli. The expression of antibodies and antibody fragments in prokaryotic cells such as E. coli is well established in the art. Expression in eukaryotic cells in culture is also available to those skilled in the art as an option for production of a binding member.

General techniques for the production of antibodies are well known to the person skilled in the field, with such methods being discussed in, for example, Kohler and Milstein (1975) Nature 256: 495-497; U.S. Pat. No. 4,376,110; Harlow and Lane, Antibodies: a Laboratory Manual, (1988) Cold Spring Harbor, the contents of which are incorporated herein by reference.

Techniques for the preparation of recombinant antibody molecules is described in the above references and also in, for example, European Patent Number 6,236,79 or European Patent Number 0,368,684, which are incorporated herein by reference.

In preferred embodiments of the invention, recombinant nucleic acids comprising an insert coding for a heavy chain variable domain and/or for a light chain variable domain of antibodies are employed. By definition such nucleic acids comprise coding single stranded nucleic acids, double stranded nucleic acids consisting of said coding nucleic acids and of complementary nucleic acids thereto, or these complementary (single stranded) nucleic acids themselves.

Furthermore, nucleic acids encoding a heavy chain variable domain and/or a light chain variable domain of antibodies can be enzymatically or chemically synthesised nucleic acids having the authentic sequence coding for a naturally-occurring heavy chain variable domain and/or for the light chain variable domain, or a mutant thereof.

Recombinant DNA technology may be used to improve the antibodies of the invention. Thus, chimeric antibodies may be constructed in order to decrease the immunogenicity thereof in diagnostic or therapeutic applications. Moreover, immunogenicity within, for example, a transgenic organism such as a pig, may be minimised, by altering the antibodies by CDR grafting in a technique analogous to humanising antibodies. Examples of such techniques are described in EP 0,239,400 to Winter. In order to reduce immunogenicity within a recipient, the invention may employ recombinant nucleic acids comprising an insert coding for a heavy chain variable domain of an antibody fused to a human constant domain. Likewise the invention concerns recombinant DNAs comprising an insert coding for a light chain variable domain of an antibody fused to a human constant domain κ or λ.

Antibodies may moreover be generated by mutagenesis of antibody genes to produce 5 artificial repertoires of antibodies. This technique allows the preparation of antibody libraries. Antibody libraries are also available commercially. Hence, the present invention advantageously employs artificial repertoires of immunoglobulins, preferably artificial ScFv repertoires, as an immunoglobulin source in order to identify binding molecules which have specificity for the epitope of the present invention.

### Antibody Selection Systems

Immunoglobulins which are able to bind to the epitope of the present invention and which accordingly may be used in the methods of the invention can be identified using any technique known to the skilled person. Such immunoglobulins may be conveniently isolated from libraries comprising artificial repertoires of immunoglobulin polypeptides. A “repertoire” refers to a set of molecules generated by random, semi-random or directed variation of one or more template molecules, at the nucleic acid level, in order to provide a multiplicity of binding specificities. Methods for generating repertoires are well characterised in the art.

Any library selection system may be used in conjunction with the invention. Selection protocols for isolating desired members of large libraries are known in the art, as typified by phage display techniques. Such systems, in which diverse peptide sequences are displayed on the surface of filamentous bacteriophage, have proven useful for creating libraries of antibody fragments (and the nucleotide sequences that encode them) for the in vitro selection and amplification of specific antibody fragments that bind a target antigen. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phage bodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straight forward.

Methods for the construction of bacteriophage antibody display libraries and lambda phage expression libraries are well known in the art (for example, McCafferty et al. (1990) Nature 348 552-554. One particularly advantageous approach has been the use of scFv phage-libraries (see for example Huston et al., 1988, Proc. Natl. Acad. Sci. USA).

An alternative to the use of phage or other cloned libraries is to use nucleic acid, preferably RNA, derived from the B cells of an animal which has been immunised with the selected target, e.g. the TLR2 epitope of the present invention.

Isolation of V-region and C-region mRNA permits antibody fragments, such as Fab or Fv, to be expressed intracellularly. Briefly, RNA is isolated from the B cells of an immunised animal, for example from the spleen of an immunised mouse or the circulating B cells of a llama, and PCR primers used to amplify VH and VL cDNA selectively from the RNA pool. The VH and VL sequences thus obtained are joined to make scFv antibodies. PCR primer sequences may be based on published VH and VL sequences.

### Peptidomimetics

Peptide analogues, such as peptidomimetics or peptide mimetics are non-peptide compounds with properties representative of a template peptide. Such peptide analogues are typically developed using computerised molecular modelling. Peptidomimetics which are structurally similar to peptides which have affinity and binding specificity to the TLR2 binding epitope of the present invention may be used to mediate similar diagnostic, prophylactic and therapeutic effects.

Peptidomimetics are typically structurally similar to a template peptide, but have one or more peptide linkages replaced by an alternative linkage, by methods which are well known in the art. For example, a peptide which has a binding specificity for the TLR2 epitope of the invention may be modified such that it comprises amide bond replacement, incorporation of non peptide moieties, or backbone cyclisation. Suitably if cysteine is present the thiol of this residue is capped to prevent damage of the free sulphate group. A peptide may further be modified from the natural sequence to protect the peptides from protease attack.

Suitably a peptide of and for use in the present invention may be further modified using at least one of C and/or N-terminal capping, and/or cysteine residue capping.

Suitably, a peptide of and for use in the present invention may be capped at the N terminal residue with an acetyl group. Suitably, a peptide of and for use in the present invention may be capped at the C terminal with an amide group. Suitably, the thiol groups of cysteines are capped with acetamido methyl groups.

### Sequence Homology

The discontinuous, conformational epitope of the present invention is derived from amino acid sequences present at both the C-terminal region and N-terminal region of the human TLR2 sequence as defined in SEQ ID NO:1. The amino acid sequences which are implicated in the epitope may be defined in SEQ ID NO:2, 3, 4 or 5.

The invention extends to the use of the peptide which have been determined as contributing the epitope, either alone, or in combination, for use in binding to Toll-like Receptor ligands. As such, the invention extends to polypeptide fragments of the amino acid of SEQ ID NO:2, 3, 4 and 5 of varying lengths, wherein the fragments define a binding epitope according to the present invention, which when bound by a ligand with a specific binding affinity, results in an inhibition of TLR2 mediated signalling regardless of whether TLR2 complexes with TLR1 or TLR6 to form a heterodimer.

The epitope may be provided in an isolated form in order to assist in the production of antibodies and binding fragments which have affinity and binding specificity to the identified binding epitope. Accordingly, the present invention extends to naturally occurring fragments and variants as well as derived variants of a polypeptide having the amino acid sequence of SEQ ID NO:2, 3, 4 and 5.

A “variant” of a polypeptide having the amino acid sequence of SEQ ID NO:2, 3, 4 or 5 means a polypeptide substantially homologous to a polypeptide having the amino acid sequence of SEQ ID NO: 2, 3, 4 or 5, but which has an amino acid sequence different from that of the polypeptide having the amino acid sequence of SEQ ID NO: 2, 3, 4 or 5 because of one or more deletions, insertions, or substitutions. The variant has an amino acid sequence that preferably is at least 80% identical to the polypeptide having the amino acid sequence of SEQ ID NO: 2, 3, 4 or 5, most preferably at least 90% identical. The percent identity may be determined, for example, by comparing sequence information using the GAP computer program, version 6.0 described by Devereux et al. (Nucl. Acids Res. 12:387, 1984) and available from the University of Wisconsin Genetics Computer Group (UWGCG).

The present invention extends to peptides which are variants, derivates or homologues of a polypeptide having the amino acid sequence of SEQ ID NO: 2, 3, 4 or 5, such peptides may have a sequence which has at least about 30%, or 40%, or 50%, or 60%, or 70%, or 75%, or 80%, or 85%, or 90%, 95%, 98% or 99% homology to the sequence of a polypeptide having the amino acid sequence of SEQ ID NO: 2, 3, 4 or 5. Thus, a peptide fragment of any one of the peptides of the invention may include 1, 2, 3, 4, 5 or greater than 5 amino acid alterations.

Moreover, or in addition, the peptide may consist of a truncated version of a polypeptide having the amino acid sequence of SEQ ID NO: 2, 3, 4 or 5 which has been truncated by 1, 2, 3, 4 or 5 amino acids.

A given amino acid may be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

Similarly, the DNAs of the invention include variants that differ from a native DNA sequence because of one or more deletions, insertions or substitutions, but that encode a biologically active polypeptide.

Expression, isolation and purification of polypeptides defining the epitope of the invention and fragments thereof may be accomplished by any suitable technique.

A method for producing polypeptides comprises culturing host cells transformed with a recombinant expression vector encoding a polypeptide having the amino acid sequence of SEQ ID NO:2, 3, 4 or 5 under conditions that promote expression of the polypeptide, then recovering the expressed polypeptides from the culture. The skilled man will recognise that the procedure for purifying the expressed polypeptides will vary according to such factors as the type of host cells employed, and whether the polypeptide is intracellular, membrane-bound or a soluble form that is secreted from the host cell.

Any suitable expression system may be employed. The vectors include a DNA encoding a polypeptide or fragment of the invention, operably linked to suitable transcriptional or translational regulatory nucleotide sequences, such as those derived from a mammalian, avian, microbial, viral, bacterial, or insect gene. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired (E. coli) host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide during translation, but allows secretion of polypeptide from the cell.

Suitable host cells for expression of polypeptides include higher eukaryotic cells and yeast. Prokaryotic systems are also suitable. Mammalian cells, and in particular CHO cells are particularly preferred for use as host cells. Appropriate cloning and expression vectors for use with mammalian, prokaryotic, yeast, fungal and insect cellular hosts are described, for example, in Pouwels et al. Cloning Vectors: A Laboratory Manual, Elsevier, N.Y., (1986) (ISBN 0444904018).

### Administration

The monoclonal antibody or fusion protein of the present invention may be administered alone but will preferably be administered as a pharmaceutical composition, which will generally comprise a suitable pharmaceutically acceptable excipient, diluent or carrier selected depending on the intended route of administration. Examples of suitable pharmaceutical carriers include; water, glycerol, ethanol and the like.

The monoclonal antibody or fusion protein of the present invention may be administered to a patient in need of treatment via any suitable route. As detailed herein, it is preferred that the composition is administered parenterally by injection or infusion. Examples of preferred routes for parenteral administration include, but are not limited to; intravenous, intracardial, intraarterial, intraperitoneal, intramuscular, intracavity, subcutaneous, transmucosal, inhalation or transdermal.

Routes of administration may further include topical and enteral, for example, mucosal (including pulmonary), oral, nasal, rectal.

In preferred embodiments, the composition is deliverable as an injectable composition. For intravenous, intradermal or subcutaneous application, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or, Lactated Ringer's injection. Preservatives, stabilisers, buffers, antioxidants and/or other additives may be included, as required.

The composition may also be administered via microspheres, liposomes, other microparticulate delivery systems or sustained release formulations placed in certain tissues including blood.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition ISBN 0-912734-04-3 and Pharmaceutical Dosage Forms and Drug Delivery Systems; Ansel, N. C. et al. 7th Edition ISBN 0-683305-72-7, the entire disclosures of which is herein incorporated by reference.

The composition is preferably administered to an individual in a “therapeutically effective amount”, this being sufficient to show benefit to the individual to whom the composition is administered. The actual dose administered, and rate and time-course of administration, will depend on, and can be determined with due reference to, the nature and severity of the condition which is being treated, as well as factors such as the age, sex and weight of the patient to be treated and the route of administration. Further due consideration should be given to the properties of the composition, for example, its binding activity and in-vivo plasma life, the concentration of the fusion protein in the formulation, as well as the route, site and rate of delivery.

Dosage regimens can include a single administration of the composition of the invention, or multiple administrative doses of the composition. The compositions can further be administered sequentially or separately with other therapeutics and medicaments which are used for the treatment of the condition for which the fusion protein of the present invention is being administered to treat.

Examples of dosage regimens which can be administered to a subject can be selected from the group comprising, but not limited to; 1 μg/kg/day through to 20 mg/kg/day, 1 μg/kg/day through to 10 mg/kg/day, 10 μg/kg/day through to 1 mg/kg/day.

The TLR2 epitope binding compound of the present invention is preferably administered to an individual in a “therapeutically effective amount”, this being sufficient to show benefit to the individual.

The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

The nomenclature used to describe the polypeptide constituents of the fusion protein of the present invention follows the conventional practice wherein the amino group (N) is presented to the left and the carboxy group to the right of each amino acid residue.

The expression “amino acid” as used herein is intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified by methylation, amidation, acetylation or substitution with other chemical groups which can change the circulating half life without adversely affecting their biological activity.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. Furthermore, the term polypeptide extends to fragments, analogues and derivatives of a peptide, wherein said fragment, analogue or derivative retains the same biological functional activity as the peptide from which the fragment, derivative or analogue is derived

Furthermore the term “fusion protein” as used herein can also be taken to mean a fusion polypeptide, fusion peptide or the like, or may also be referred to as an immunoconjugate. The term “fusion protein” refers to a molecule in which two or more subunit molecules, typically polypeptides, are covalently or non-covalently linked.

As used herein, the term “therapeutically effective amount” means the amount of a fusion protein of the invention which is required to reduce the severity of and/or ameliorate a TLR2 mediated disease, a cancerous condition or a disease such as an autoimmune disease or a neurodegenerative disease or at least one symptom thereof.

As used herein, the term “prophylactically effective amount” relates to the amount of a composition which is required to prevent the initial onset, progression or recurrence of a TLR2 mediated or induced disease or condition, or a disease such as an autoimmune disease or a neurodegenerative disease or at least one symptom thereof in a subject following the administration of the compounds of the present invention.

As used herein, the term “treatment” and associated terms such as “treat” and “treating” means the reduction of the progression, severity and/or duration of a TLR2 mediated condition of at least one symptom thereof, wherein said reduction or amelioration results from the administration of a binding compound which has specificity for the TLR2 binding epitope of the present invention. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

As used herein, the term “subject” refers to an animal, preferably a mammal and in particular a human. In a particular embodiment, the subject is a mammal, in particular a human. The term “subject” is interchangeable with the term “patient” as used herein.

The present invention will now be described with reference to the following examples which are provided for the purpose of illustration and are not intended to be construed as being limiting on the present invention.

### EXAMPLES

### Example 1

### Anti-TLR2 Monoclonal Antibody In-Vitro Bioassay

### (i) Experiment 1—Determination of IC50

U937 cells (Human leukemic monocyte lymphoma cell line) at 2×10⁵/ml were treated with 100 ng/ml of the tri-acylated lipopeptide, Pam3CSK4, and 12 serial dilutions of an anti-TLR2 antibody OPN-301 ((OPN301), a murine IgG1 anti-TLR2 antibody (mouse Toll-like Receptor 2 (TLR2) antibody, clone T2.5, HyCult Biotechnology b.v., Cell Sciences, Canton, USA: catalogue number 1054, HM1054BULK and HM5323M17), or an isotype control mouse IgG1 (Southern Biotech, catalogue number 0102-01) antibody. After a 6 hour incubation period cell supernatants were removed and TNF-alpha concentrations were assayed by human specific ELISA (Cytokine duoset from R&D systems). IC50 experiments where repeated with lot 5323M17 and performed as least twice for reproducibility.

### Results

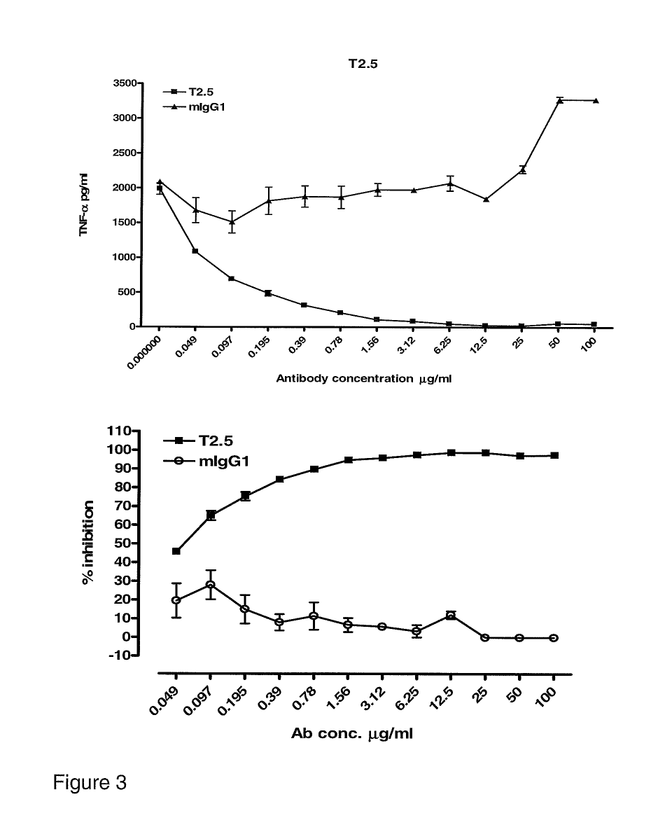

The results are shown in FIG. 3, graphs (a) and (b). FIG. 3(a) shows TNF-alpha production from U937 cells stimulated with 100 ng/ml Pam3CSK4 alone or in the presence of the anti-TLR2 monoclonal antibody OPN-301, or in the presence of a murine isotype control antibody designated mIgG1. FIG. 3(b) shows the percentage of TNF alpha inhibition of U937 cells treated with the tri-acylated lipopeptide Pam3CSK4 and an anti-TLR2 monoclonal antibody or murine IgG1 isotype control relative to Pam3CSK4 only treated cells. Supernatants were removed after 6 hours for cytokine analysis.

### (ii) Experiment 2—Specificity

U937 cells at 2×10⁵/ml were treated with a TLR2/TLR1 heterodimer agonist, specifically the tri-acylated lipopeptide Pam3CSK4, the TLR-2/TLR-6 heterodimer agonist FSL-1, and Low Molecular Weight Hyaluronic Acid (LMW HA). Further, to test specificity, 100 ng/ml of lipopolysaccharide (LPS) and serial dilutions of the anti-TLR2 monoclonal antibody OPN-301, or a murine isotype control antibody mIgG1 (Southern Biotech, catalogue number 0102-01) antibody were added. Lot 5323M17 was used to confirm inhibition of lot HM1054 in U937 cells and to determine specificity in murine cells. After a 6 hour incubation period cell supernatants were removed and TNF-alpha concentrations assayed by human specific ELISA (Cytokine duoset from R&D systems).

### Results

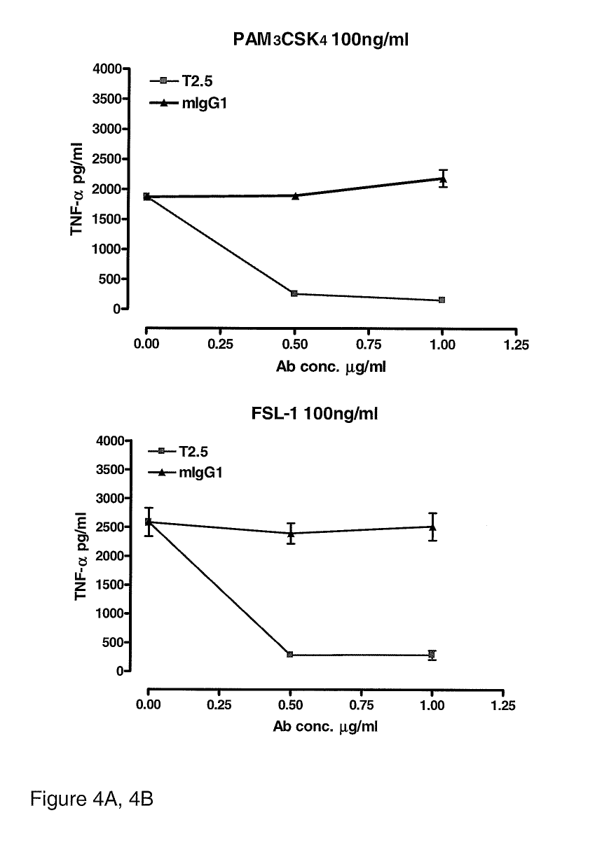

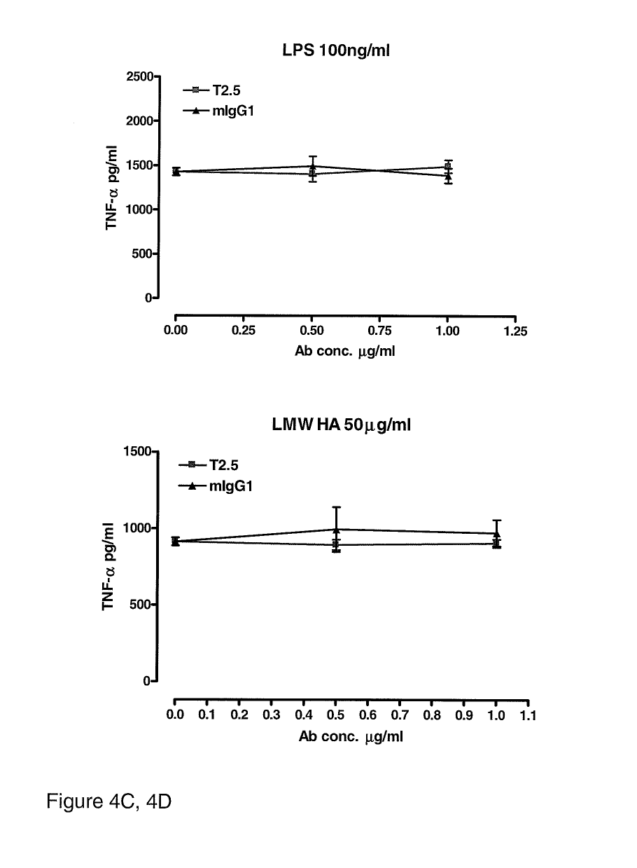

The results are shown in FIG. 4. TNF-alpha secretion from U937 cells stimulated with 100 ng/ml Pam₃CSK₄, 100 ng/ml FSL-1, 100 ng/ml LPS or 50 μg/ml LMW HA alone or in the presence of 0.5 μg/ml or 1.0 μg/ml anti-TLR2 monoclonal antibody or isotype control antibody, murine IgG1. Supernatants were removed after 6 h for cytokine analysis.

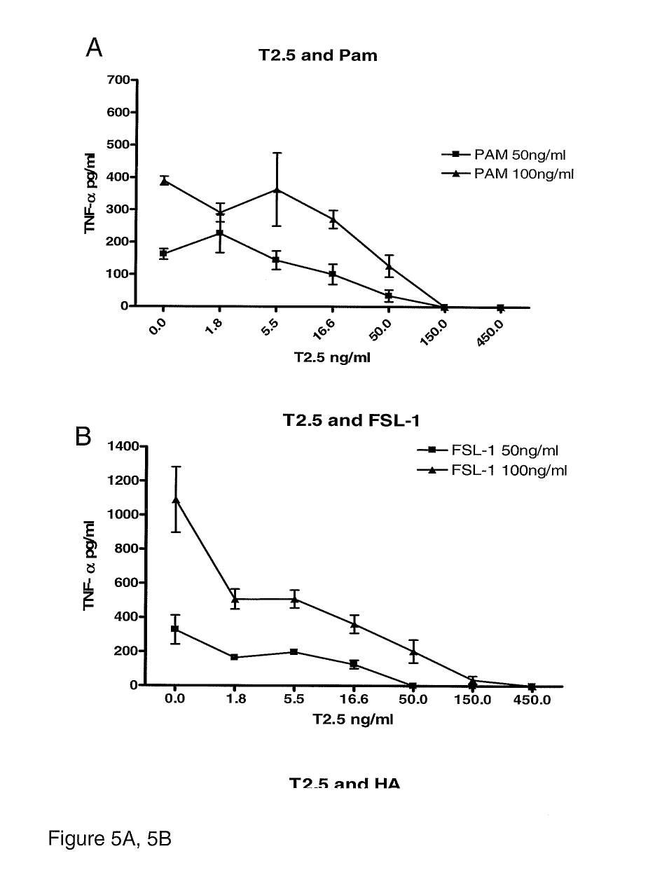

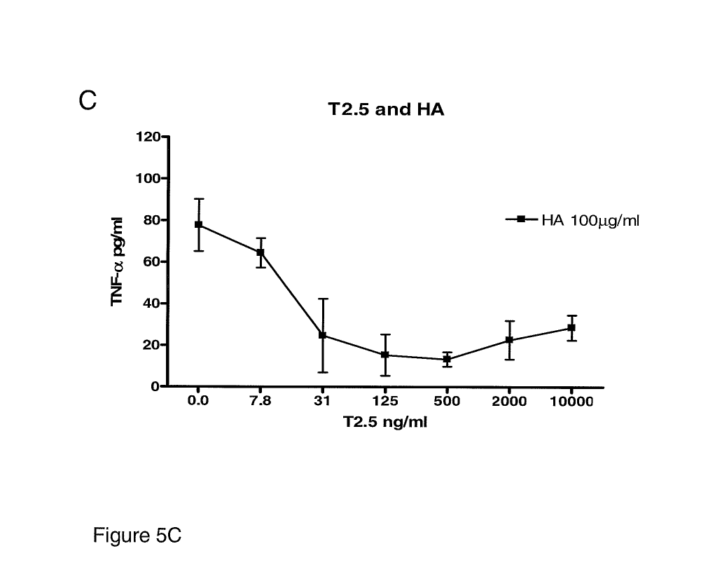

The specificity of anti-TLR2 monoclonal antibody was further assessed in a second set of experiments wherein TNF-alpha secretion from U937 cells stimulated with 50 and 100 ng/ml Pam3CSK4 and FSL-1 or 100 μg/ml LMW HA (in the presence of Polymyxin B sulphate) alone or in the presence of serial dilutions of anti-TLR2 monoclonal antibody or isotype control antibody, murine IgG1. Supernatants were removed after 6 hours for cytokine analysis. The results of these experiments are shown in FIG. 5.

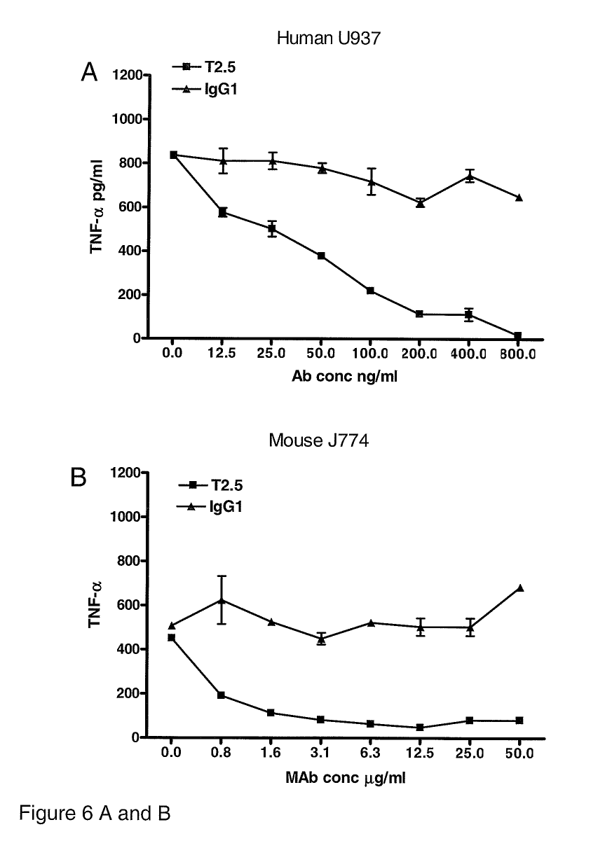

FIG. 6 shows further graphs illustrating that TNF-alpha secretion from human U937 and murine J774 cells stimulated with 100 ng/ml Pam3CSK4 alone or in the presence of serial dilutions of anti-TLR2 monoclonal antibody or isotype control antibody, murine IgG1. Supernatants were removed after 6 hours for cytokine analysis.

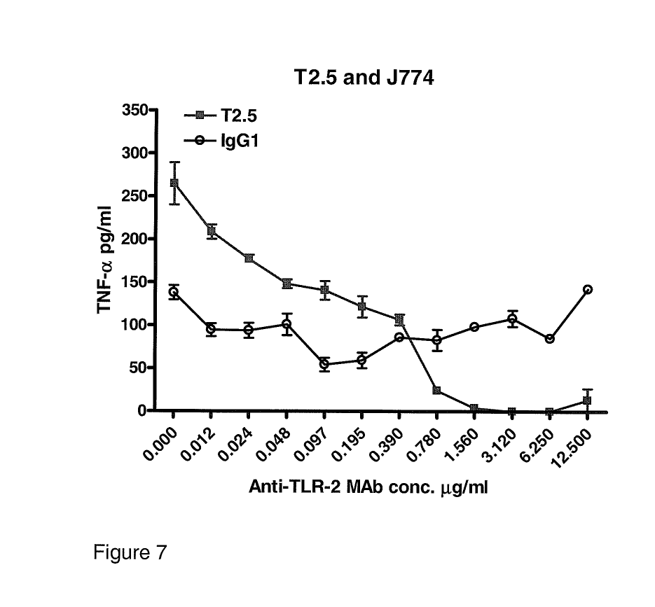

FIG. 7 shows TNF-alpha secretion murine J774 cells stimulated with 100 ng/ml Pam3CSK4 alone or in the presence of serial dilutions of anti-TLR2 monoclonal antibody or isotype control antibody, murine IgG1. Supernatants were removed after 6 hours for analysis.

(iii) Experiment 3—Ex-Vivo Human PBMC

Human PBMC from two male donors (S1 and S2) were cultured at 5×10⁵/ml and were treated with 5 concentrations of Pam3CSK4 and 6 dilutions of the anti-TLR2 monoclonal antibody OPN-301 or a murine IgG1 isotype control mouse antibody (Southern Biotech, catalogue number 0102-01). Human PBMC were isolated and purified according to SOP LAB021. After a 6 hour incubation period cell supernatants were removed and TNF-alpha, IL-12p40, IL-1beta, MIP-1alpha, IL-8 and IL-6 concentrations were assayed by human specific ELISA (Cytokine duosets from R&D systems).

### Results

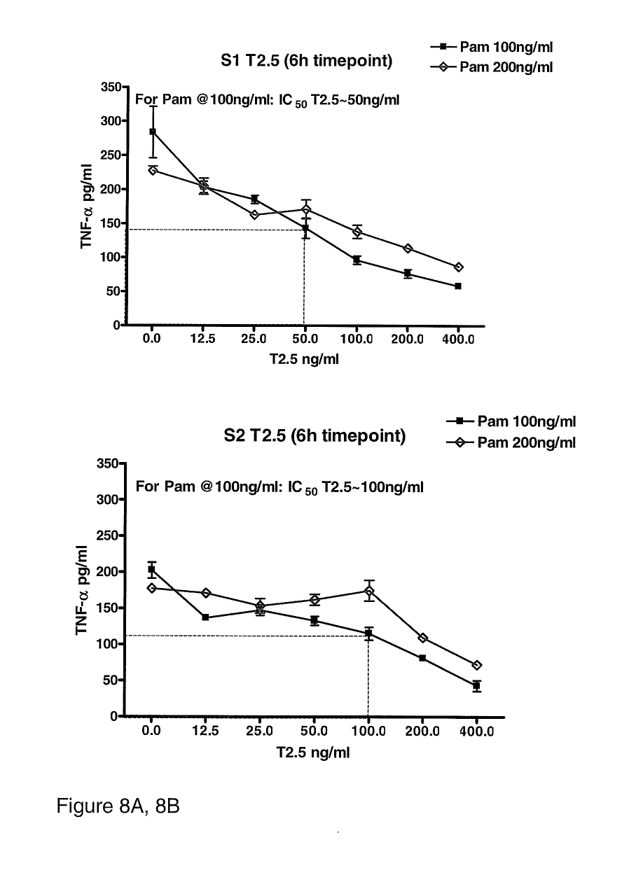

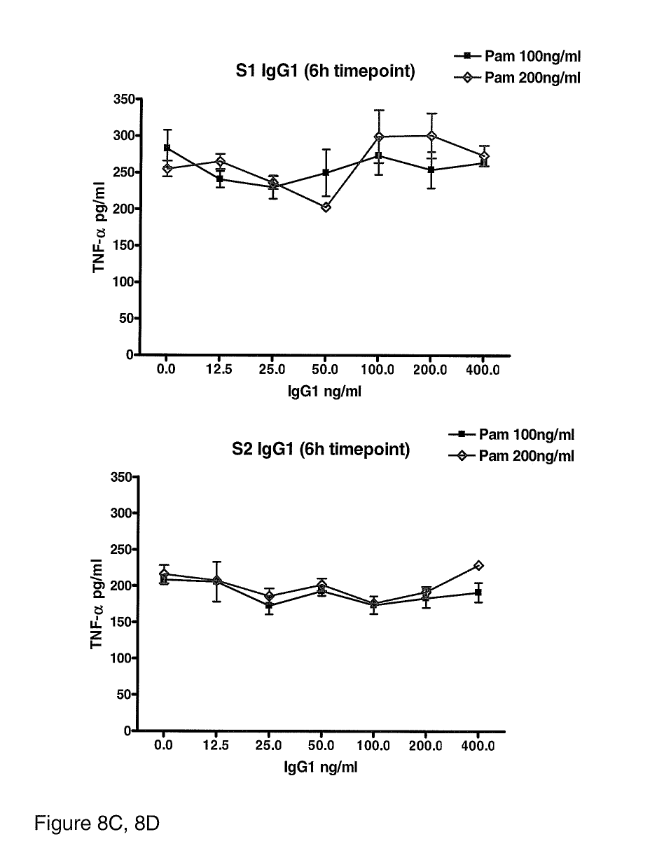

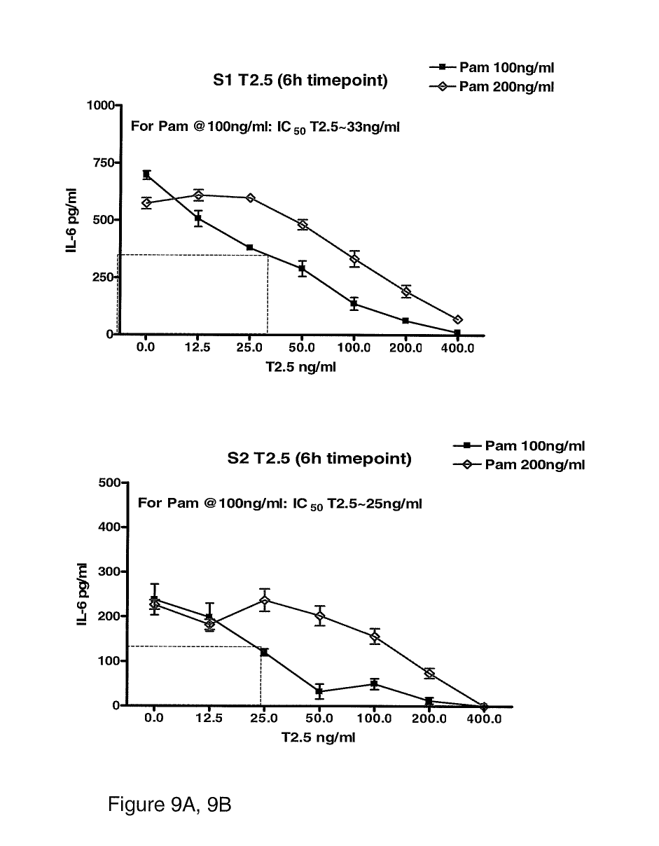

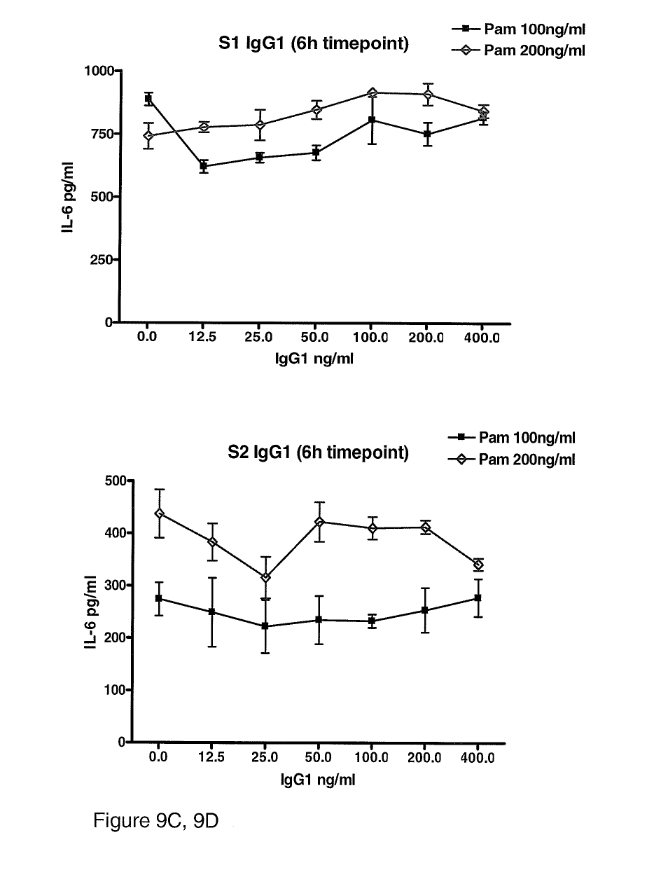

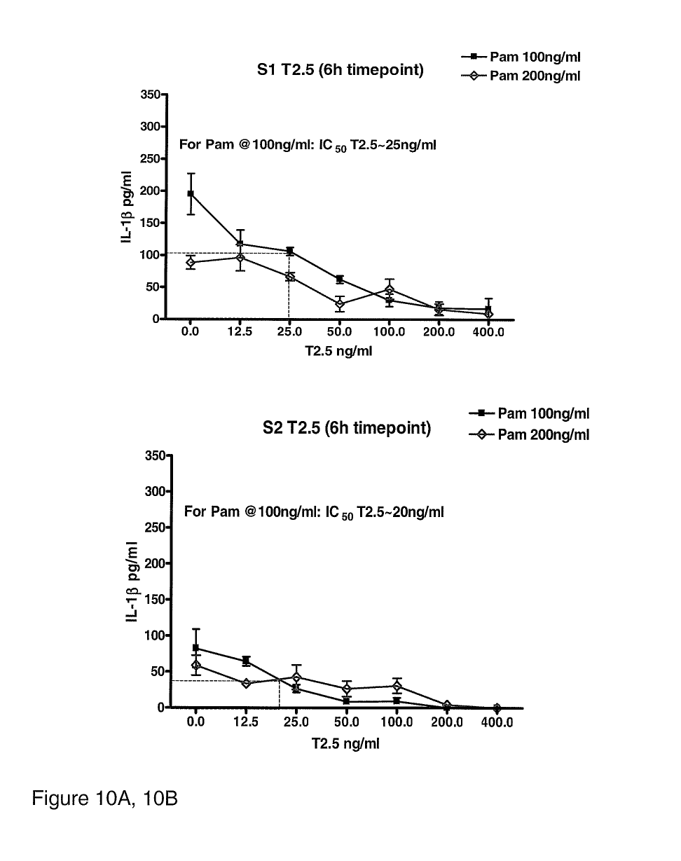

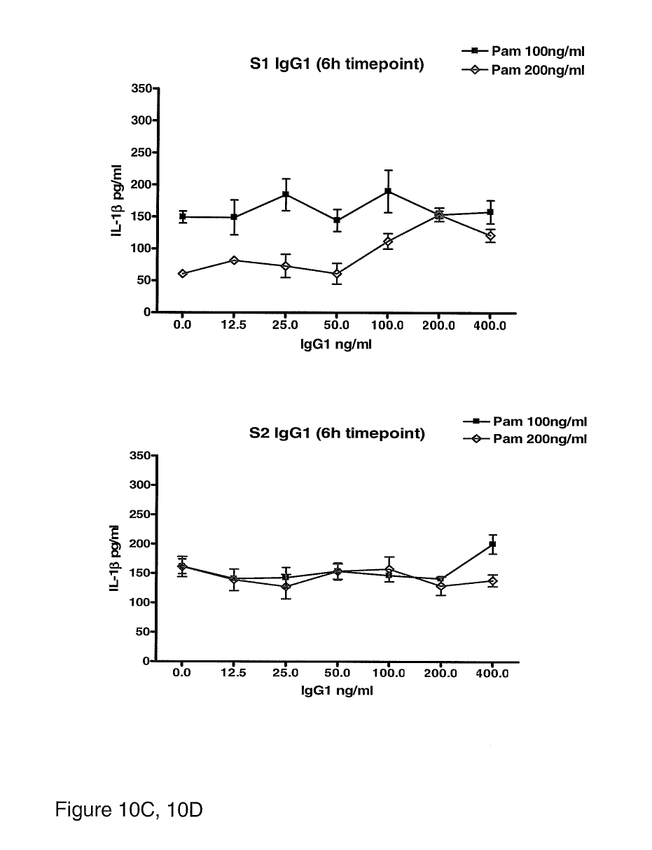

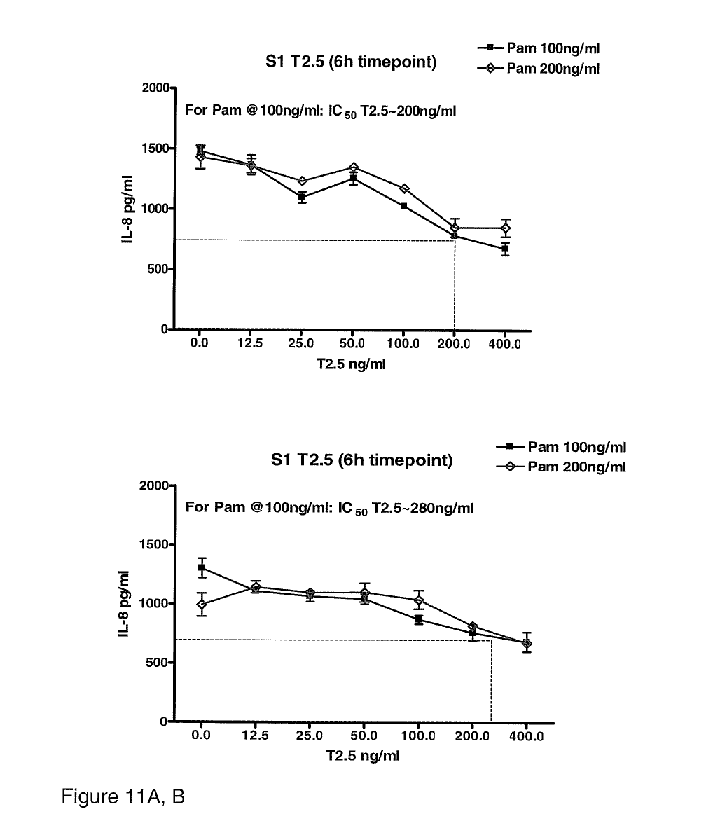

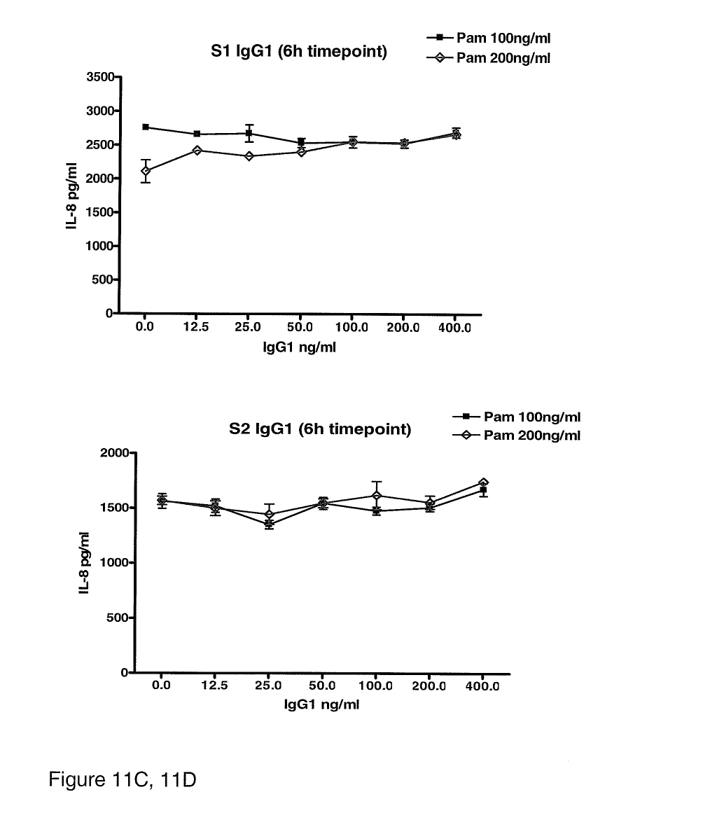

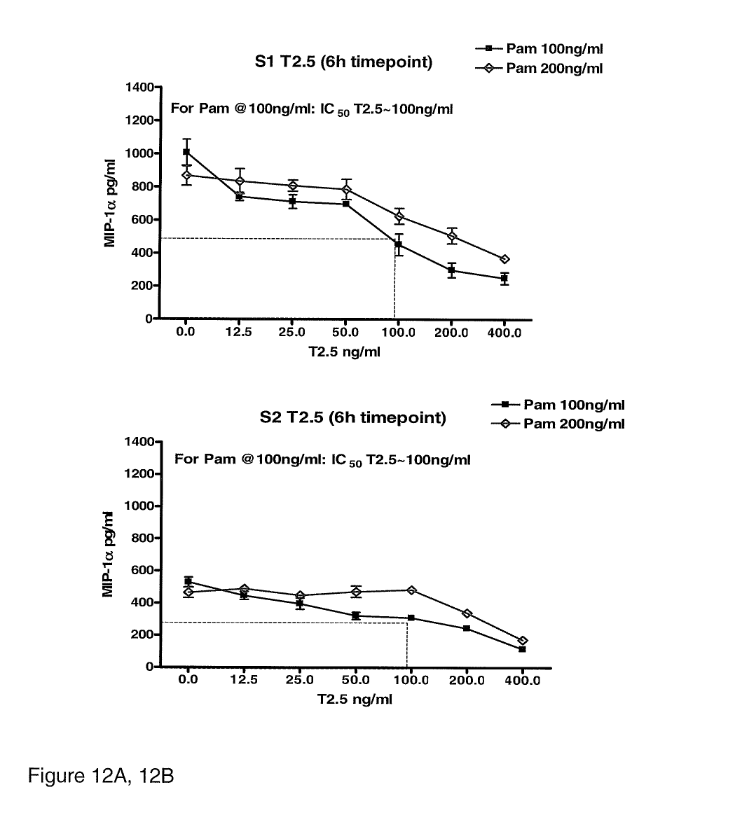

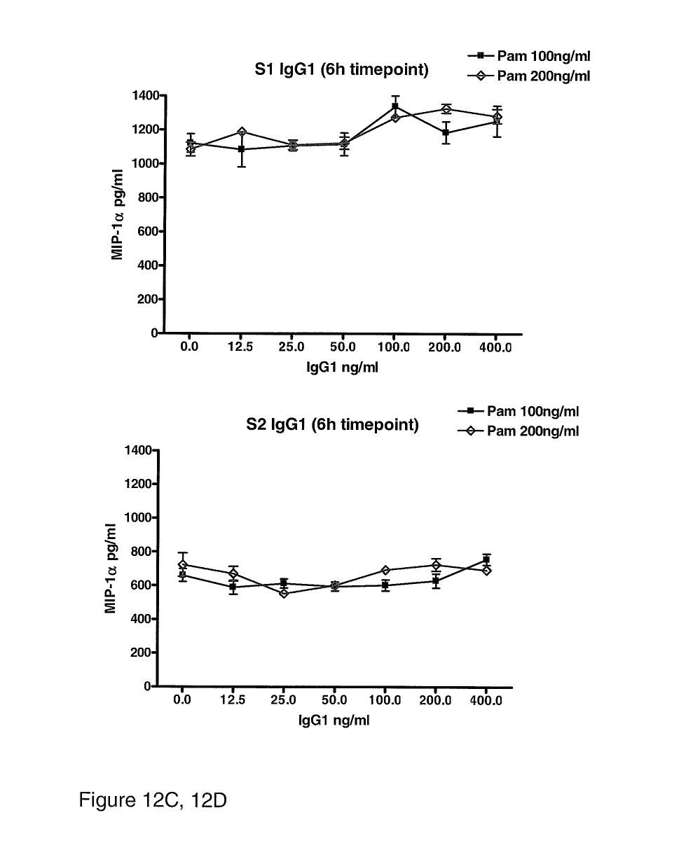

The results are shown in FIG. 8 (TNF-alpha), FIG. 9 (IL-6), FIG. 10 (II-1beta), FIG. 11 (IL-8) and FIG. 12 (MIP-1alpha). In each of the above FIGS. 8 to 12), graphs (a) and (b) show the results of samples S1 and S2 to OPN-301 monoclonal antibody, while graphs (c) and (d) show the results relating to the murine IgG1 isotype control monoclonal antibody. Supernatants were negative for IL-12p40 (data not shown).

### Conclusion

The OPN-301 anti-TLR2 monoclonal antibody is specific for TLR2 and has an IC50 of approximately 30 ng/ml when tested against 100 ng/ml Pam₃CSK₄. However, in initial experiments the OPN-301 monoclonal antibody was not observed to inhibit low molecular weight hyaluronic acid (LMW HA) mediated TNF-alpha secretion from U937 cells. LMW HA is thought to be an important mediator of pro-inflammatory cytokine secretion in certain auto-immune diseases such as Rheumatoid arthritis and to mediate these inflammatory responses through TLR2. However, when these assays were repeated in the presence of the LPS chelating agent Polymyxin B sulphate, anti-TLR2 antibody was observed to inhibit LMW HA derived TNF-alpha suggesting the failure to observe inhibition previously was due to interference with contaminating LPS. Anti-TLR2 antibody was not observed to inhibit the response mediated by the TLR4 agonist LPS. In addition the OPN-301 monoclonal antibody was shown to inhibit TNF-alpha secretion from murine J774 cells and a wide range of cytokines and chemokines from human PBMC.

### Definitions and Reagents/Tests Performed.

Pam3CSK4 is a triacylated lipopeptide which acts as a synthetic agonist to the TLR1/TLR2 heterodimer (Invivogen, catalogue number tlrl-pms). FSL-1 is a diacylated Mycoplasma derived TLR2/TLR6 heterodimer agonist (Invivogen, catalogue number Url-ftl). LPS (lipopolysaccharide) is a TLR-4 agonist (ALEXIS, E coli LPS, catalogue number 581-007-L002, lot L12922. LPS was sonicated prior to use).

Human CD14 was purchased form R&D system, Catalogue number 383-CD. The TLR2 antibody use was OPN-301. Control murine IgG1 was purchased from Southern Biotech, catalogue number 0102-01. Low Weight Hyaluronic Acid (LMW HA) was purchased from Calbiochem, Catalogue number 385902.

### Example 2

### TLR-2 Fusion Protein and Anti-TLR2 Monoclonal Antibody Binding Study

U937 cells at 2×10⁵ cells/ml were incubated with 9 different concentrations of the TLR2/TLR1 heterodimer agonist Pam3CSK4. Cells were stimulated for 6 hours with the TLR2 agonists in the presence or absence of serially diluted native TLR2-Fc and/or control murine IgG2a (R&D, catalogue number MAB003). In addition, soluble human CD14 (R&D systems, catalogue number 383-CD) was added at a final concentration of 100 ng/ml. The TLR-2-Fc fusion protein in the presence of hCD14 and TLR2 agonist was pre-incubated for 15 minutes at room temperature prior to the addition of cells.

Murine TLR2-Fc (mTLR2Fc) is a fusion of the extracellular domain of murine TLR2 receptor and the Fc domain of mouse IgG2a. Protein batch RP002, stock 980 μg/ml used for all assays.

### Results

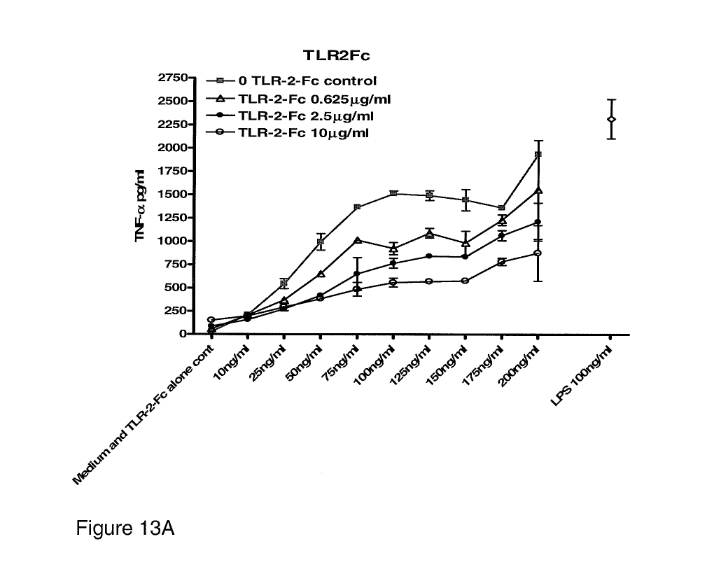

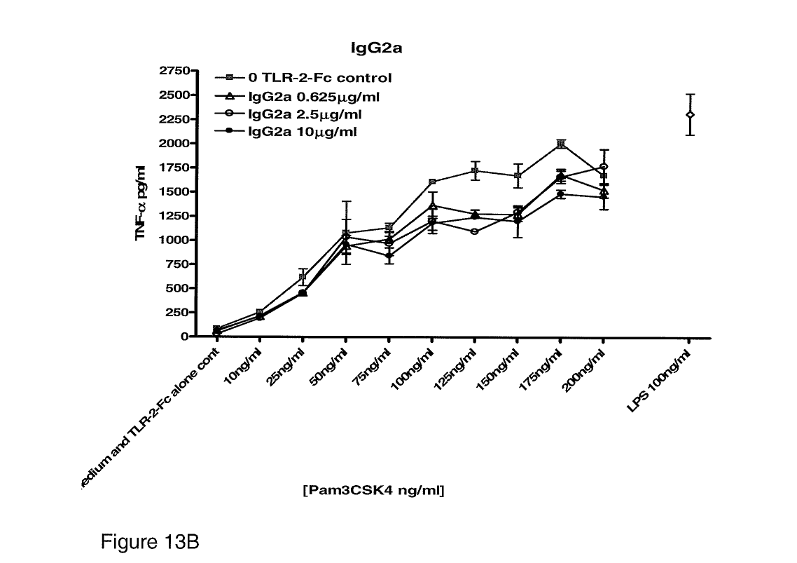

Murine TLR2Fc inhibits TNF-alpha production from TLR2 agonist stimulated cells. TNF-alpha production from U937 cells stimulated with Pam3CSK4 and 100 ng/ml recombinant human CD14 in the presence (a) mTLR2Fc (FIG. 13(a), or (b) murine IgG2a (FIG. 13(b)). U937 cells were stimulated for 6 hours with Pam3CSK4/CD14 alone (solid square) or Pam3CSK4 in the presence of TLR2 Fc fusion protein (open triangle, open circle, solid circle). LPS at 100 ng/ml was used as a positive control. The results are illustrated in FIG. 13(a) for the mTLR2Fc and in FIG. 13(b) for the IgG2a.

### Example 3

### Human TLR2 Epitope Mutation Studies

Three mutant forms of the human TLR2 receptor were made by site directed mutagenesis of the cysteine amino acid residues present at amino acid residue positions 30, 36 and 539 of the human Toll-like Receptor, the full length amino acid sequence of which is provided herewith as SEQ ID NO:4.

Specifically, these cysteine residues at positions 30, 36 and 539 of hTLR2 were substituted by serine residues using Quikchange XL mutagenesis kit (Stratagene) using primers listed in Table 1. pUNO-hTLR2 plasmid (Invivogen) served as template.

TABLE 1
primers for mutagenesis of human Toll-like
Receptor 2

| | |
|---|---|
| Cys30Ser | 5′-aggcttctctgtcttctgacc |
| (Substitution of cysteine | gcaatggtatc-3′ |
| residue at position 30 of | (SEQ ID NO: 6) |
| human TLR2 to serine | 5′-gataccattgcggtcagaaga |
| reside) | cagagaagcctg-3′ |
| | (SEQ ID NO: 7) |
| Cys36Ser | 5′-gaccgcaatggtatctgtaag |
| (Substitution of cysteine | ggcagctca-3′ |
| residue at position 36 of | (SEQ ID NO: 8) |
| human TLR2 to serine | 5′-tgagctgcccttacagatacc |
| reside) | attgcggtc-3′ |
| | (SEQ ID NO: 9) |
| Cys539Ser | 5′-aacttcatttgctcctctgaa |
| (Substitution of cysteine | ttcctctccttc-3′ |
| residue at position 539 | (SEQ ID NO: 10) |
| of human TLR2 to serine | 5′-gaaggagaggaattcagagga |
| reside) | gcaaatgaagtt-3′ |
| | (SEQ ID NO: 11) |

The expression of wild type and mutant forms of TLR2 was confirmed by western blotting.

Experiment 1

FACS Analysis

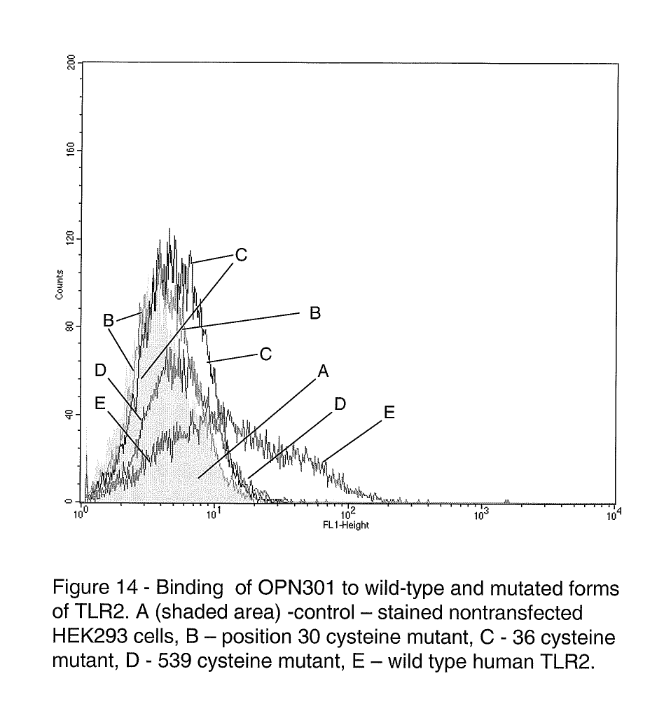

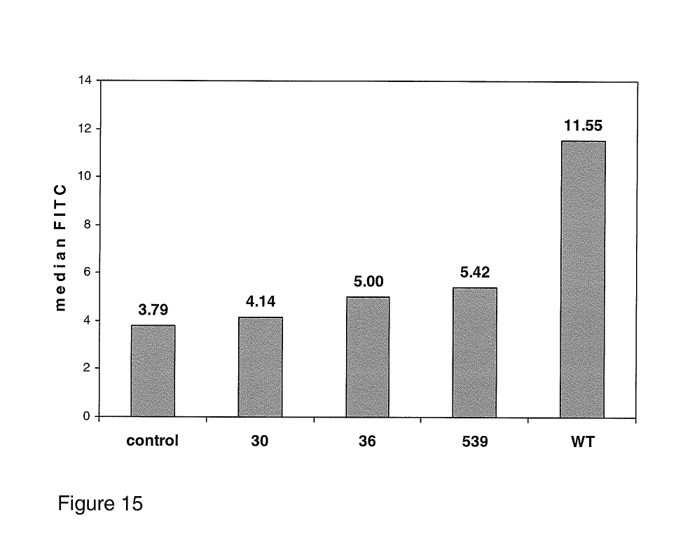

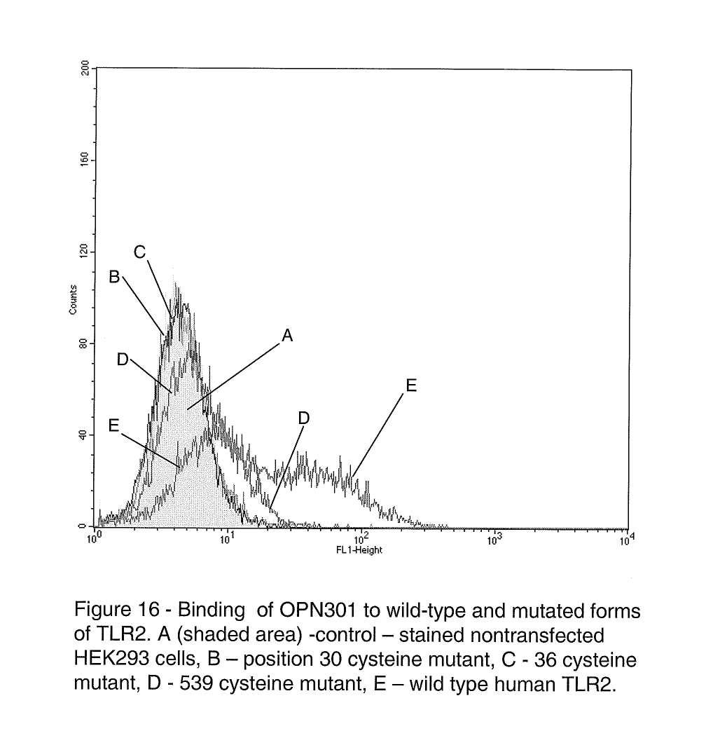

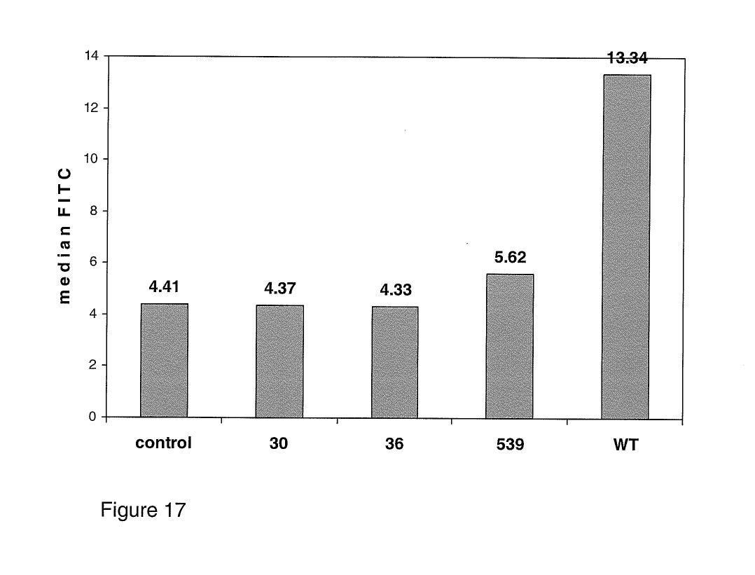

2×10⁶ HEK cells were plated in a 100 mm dish. Cells were transfected with 5 μg of pUNO vector expressing either wild type (wt) Human Toll-like Receptor 2 (hTLR2) or one of the mutant versions of TLR2 made by site directed mutagenesis of the cysteine amino acid residues present at amino acid residue positions 30, 36 and 539 as described hereinbefore. Cells were harvested 48 hours post transfection using a trypsin/edta procedure. 1×10⁶ cells were resuspended in 100 μl of FACS buffer (PBS, 2% BSA and 0.05% sodium azide). Cells were stained with OPN-301 antibody (1 μg/ml), washed and incubated with anti-mouse FITC antibody (1:200, Jackson Immunoresearch). Cells were washed with FACS buffer, resuspended in sheath fluid and analyzed on FACScalibur machine. Binding of the wild type, or mutated forms of TLR2 was assessed by FACS analysis, with the results being compared to non-mutated stably transfected HEK TLR2 cells. The results are shown in FIGS. 14 and 15 (experiment 1) and FIGS. 16 and 17 (experiment 2).

In FIGS. 14 and 16, cells were transfected with wild type or mutant forms of TLR2 and stained with OPN-301 and anti-mouse FITC. Yellow fill represents stained mock transfected cells. The lines labelled B, C, D and E represent the Cys30Ser, Cys36Ser, Cys539Ser and wild type transfectants, respectively. FIGS. 15 and 17 meanwhile show the median of FITC fluorescence intensity of wild-type and mutant forms of TLR2. Control represents stained mock-transfected cells. Functional responses between the transfected cells were also compared as described below.

Experiment 2

NF-κB Luciferase Assay

HEK cells were seeded at 2×10⁵ cell/ml in a 96 well plate and incubated overnight. For dose response experiments, cells were transfected with 1 ng, 10 ng and 100 ng of pUNO vector expressing either wild type human Toll-like receptor 2 (hTLR2) or a mutant versions of TLR2 using Genejuice reagent (Novagen). The amount of total DNA was kept constant with the addition of empty vector. For antibody blocking experiments, cells were transfected with 50 ng of wild type or mutant TLR2. Further, 80 ng of expression vector with firefly luciferase under control of NF-κB promoter and 40 ng of vector constitutively expressing Renilla luciferase was added. Cells were incubated overnight and then stimulated either with 20 ng/ml Pam3CSK4 or 10⁷ cells/ml HKLM. OPN301 antibody and an isotype control antibody (murine IgG1 as previous) were added at a final concentration of 1 μg/ml.

Cells were stimulated for 20 hours, luciferase activity was measured and results were normalised to Renilla luciferase read.

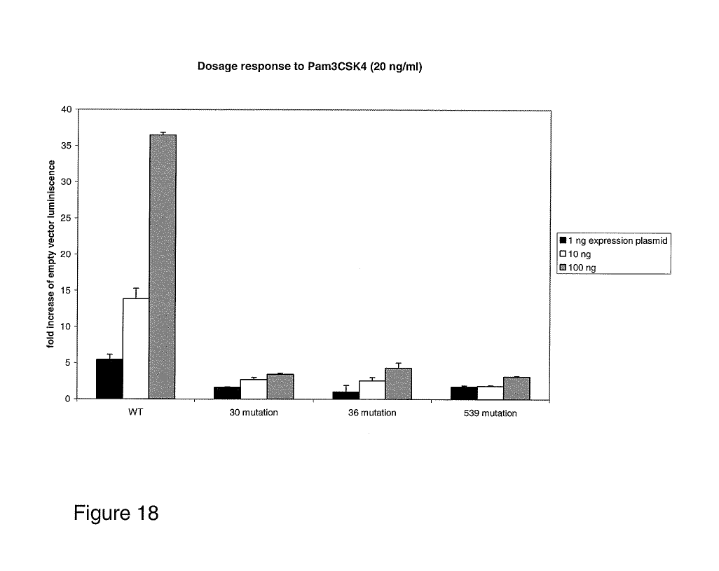

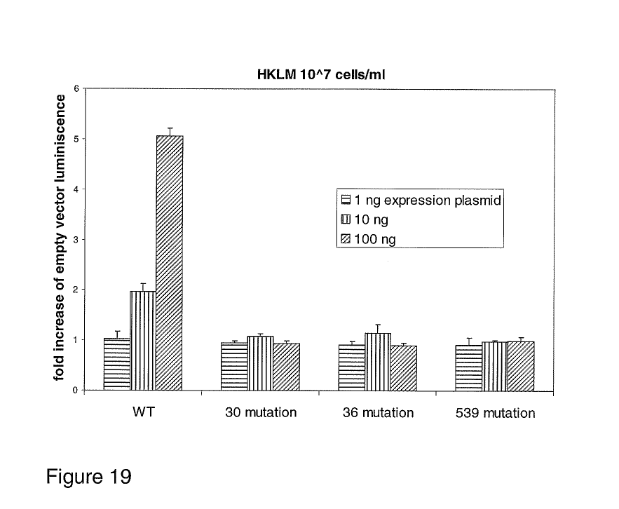

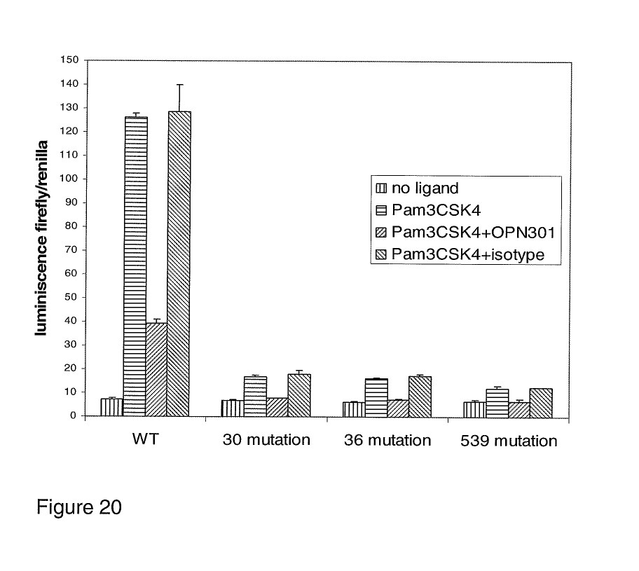

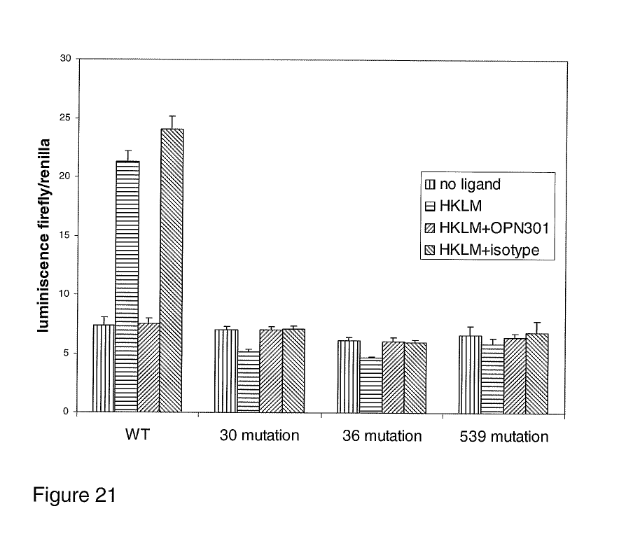

The results are shown in FIGS. 18 and 19 which show the response to different dosages of expression plasmid DNA, and FIGS. 20 and 21 which show the blocking activity of the TLR2 antagonistic antibody OPN-301. In FIGS. 18 and 19, cells were transfected with 1 ng, 10 ng and 100 ng of pUNO vector expressing wilt type and mutant versions of hTLR2 as described in material and methods. In FIG. 18, cells were stimulated with Pam3CSK4 (20 ng/ml). In FIG. 19 cells were stimulated with HKLM (10⁷ cells/ml).

In FIGS. 20 and 21 cells were transfected with 50 ng of pUNO vector expressing wild type (wt) and mutant versions of hTLR2 as described above. OPN-301 monoclonal antibody and murine IgG1 isotype control antibody were added 30 minutes before stimulation where indicated. In FIG. 20, cells were stimulated with Pam3CSK4 (20 ng/ml). In FIG. 21, cells were stimulated with Pam3CSK4 (20 ng/ml).

The results of the experiments detailed in this example indicate that the OPN-301 antibody does not bind to any of the mutated forms of TLR2. This suggests that the site-directed mutagenesis, results in a mutation which affects binding of the antagonistic antibody TLR2. Without wishing to be bound by theory, the inventors predict, based on this information, that the antagonistic anti-TLR2 antibody, OPN-301 cannot bind to the mutated forms of TLR2 described herein, this suggesting that the mutations affect the binding site on TLR2 to which OPN-301 binds. Furthermore, OPN-301 inhibits stimulation of the TLR1/TLR2 heterodimer Pam3CSK4 stimulation of wild type TLR2, this supporting the concept of OPN-301 binding to a discontinuous epitope on human TLR2.

In summary, the results show that mutation of cysteine residues identified as being present at the N terminal and C terminal regions of human TLR2 have a fundamental affect on TLR2 activity as mutation of these residues prevents binding of a TLR2 antagonistic antibody. Without wishing to be bound by theory, the inventors predict that the conserved N terminal and C terminal cysteine residues may be forming local disulfide bonds, the presence of which is important for the overall folding of the epitope to allow the binding of antagonistic binding members for use in suppression TLR2 function.

### Example 4

### Determination of TLR2 Binding Epitope Using Linear Scanning Technique

The OPN-301 monoclonal antibody was examined in order to identify the epitope present on Toll-like Receptor 2 to which it binds. 769 overlapping linear 15-mer peptides derived from the human Toll-like Receptor 2 (as defined in SEQ ID NO:4) were prepared. These 15-mer peptides overlapped to the extent that there was a 1 amino acid frameshift in each successive peptide. Hence, the first 15-mer peptide had the amino acid sequence MPHTLWMVWVLGVII, the second PHTLWMVWVLGVIIS, the third HTLWMVWVLGVIISL and so on and so forth. The 15-mer peptides covered all of the extracellular, transmembrane and extracellular regions of human TLR2.

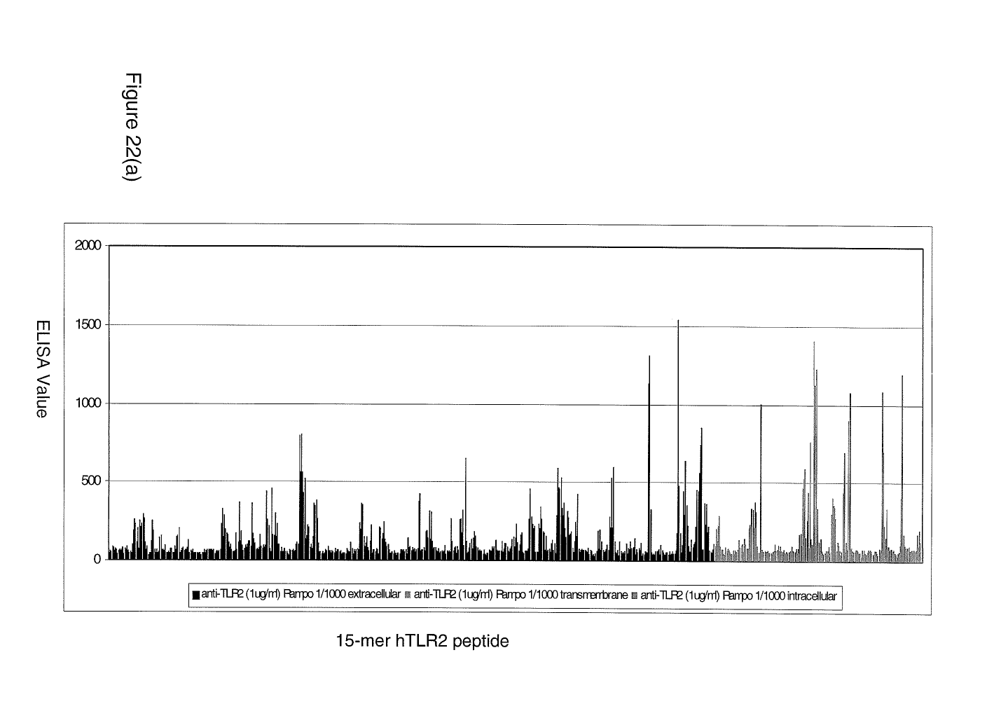

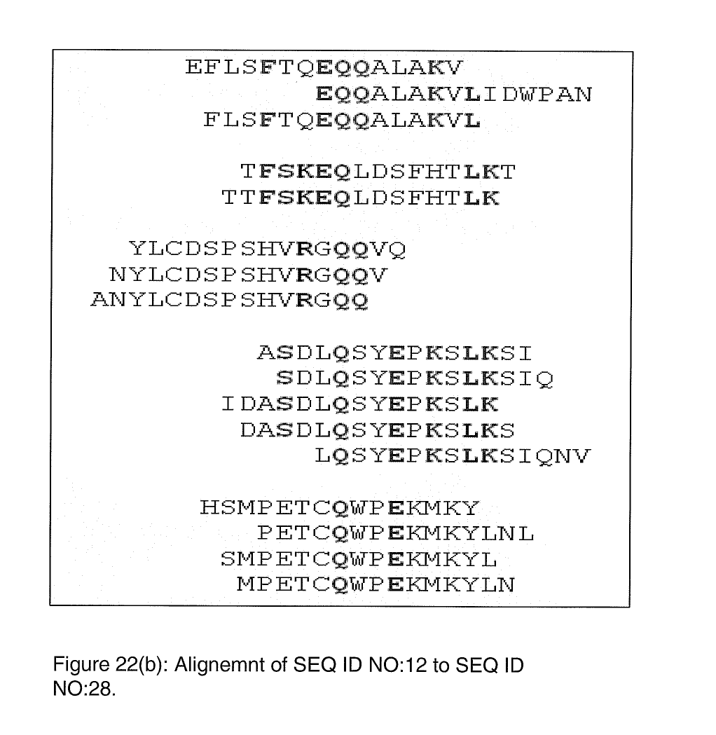

The OPN-301 monoclonal antibody was tested at a concentration of 1 ug/ml. The affinity of binding to each of the 15-mer peptides is shown in FIG. 22(a). Binding to peptides in the extracellular domain are shown by the solid black peaks. The peptides of the transmembrane region are shown in the peak region coloured with the horizontal lines, while the peptides from the intracellular domain are shown by the peak region coloured with the horizontal lines.

The results of this experiment show that the epitope to which OPN-301 binds is not likely to be a linear epitope, as the interpretation of the peaks of FIG. 22(a) do not suggest the presence of a linear epitope, this being defined by interpreting the data to show at least three consecutive overlapping linear peptides that show ELISA-values of more than 2000, with all other peptides giving values less than 100. FIG. 22(b) shows the sequence of the peptides relevant to the peaks of FIG. 22(a) which showed the highest binding affinity to OPN-301. The peptides listed in FIG. 22(b) are accorded SEQ ID NO:12 to SEQ ID NO:28 respectively. SEQ ID NO:12 being the linear peptide sequence having the amino acid sequence EFLSFTQEQQALAKV, the sequence listed under it being SEQ ID NO:13, having the amino acid sequence EQQALAKVLIDWPAN and so on and so forth for the remaining peptides in the list which are designated SEQ ID NO:15 to SEQ ID NO:28.

Thus, without wishing to be bound by theory, the inventors concluded, based on the information provided in this experiment that the epitope to which OPN-301 binds is likely to be a non-linear epitope.

### Example 5

### Determination of TLR2 Antagonistic Binding Epitope

Further characterisation of the epitope which is bound by TLR2 antagonistic antibodies, such as OPN-301 was determined using a commercially available epitope mapping technique celled CLIPS (chemically linked immunogenic peptides on scaffolding) (Pepscan Presto, the Netherlands). This technique is disclosed in Timmerman et al. “Rapid and Quantative Cyclization of Multiple Peptide”, ChemBioChem 2005, 6 pages 1-5. The CLIPS technique is based on a technique which uses spatially defined peptides to mimic complex protein structures. The interaction of proteins is mediated by surface-exposed domains consisting of one or more loops. Linear peptides are usually structurally undefined, which makes them poor mimics of protein domains. The technique therefore involves the production of one or more peptides in the form of a spatially defined construct. Due to their rigid structure these molecules behave as synthetic protein domains. This makes them very well applicable to identify and reconstructs complex protein interaction sites.

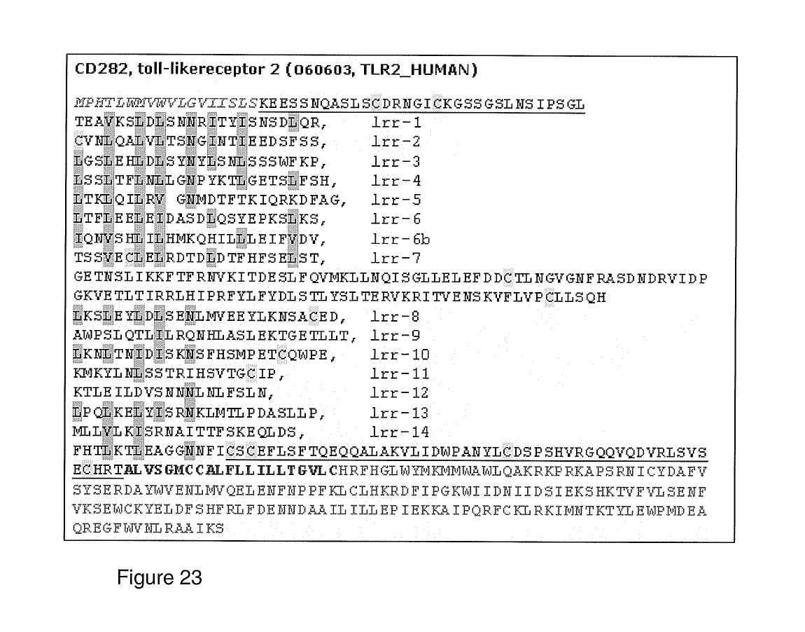

The CLIPS process commenced with 3557 different peptides being designed and synthesised, based on human TLR2. These peptides were designed with the aim of mimicking possible conformational and discontinuous epitopes. In particular, there was emphasis towards the recognition of the disulphide rich regions of the N and C terminus portions of TLR2, as the inventors had previously identified that these regions may surprisingly be involved in antibody binding. Further peptides aimed to reconstruct the 14 different LRR-folds. FIG. 23 illustrates the amino acid sequence of human TLR2 which has been broken up to show the N terminal region, the 14 leucine rich repeat domains (LRR) and the C terminal extracellular domain. The three dimensional crystal structure of TLR2 has not been definitively defined in the art. However, FIG. 23 shows that from the amino acid sequence of TLR2, it can be defined that TLR2 has 14 leucine rich repeats, with an N-terminal and C-terminal cysteine rich region. The 14 leucine rich repeats (LRRs) contain another region in between LRR 1 to 7 and LRR 8 to 14.

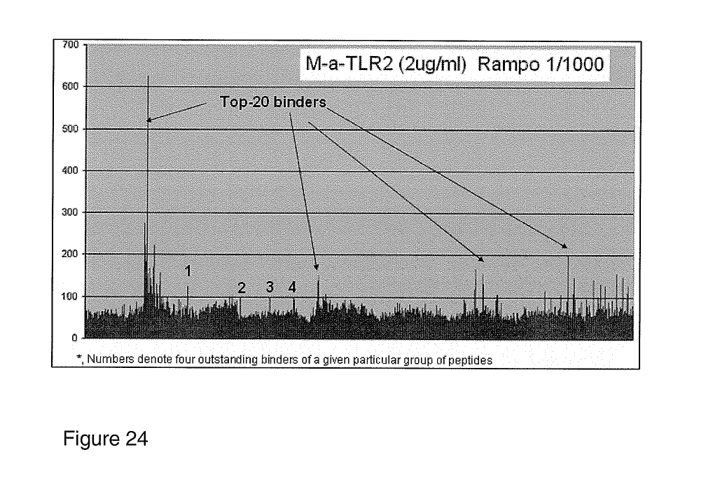

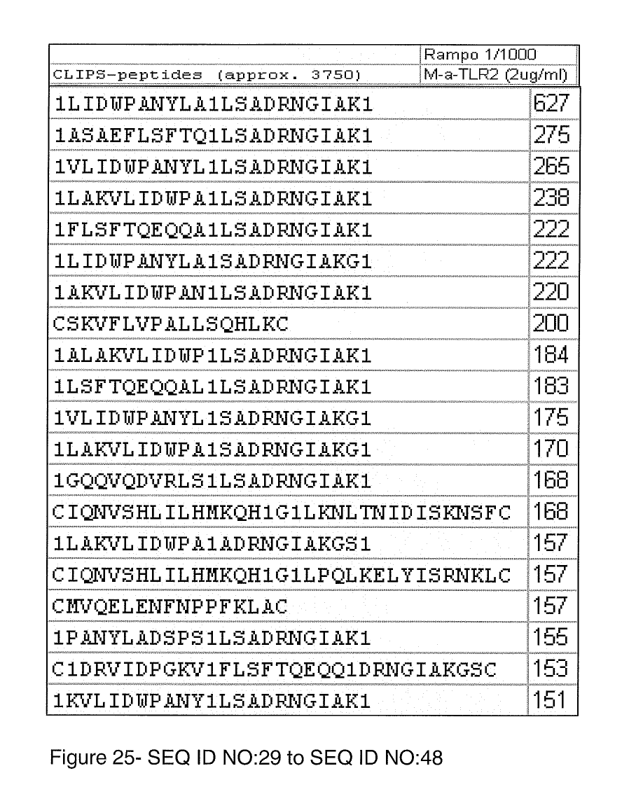

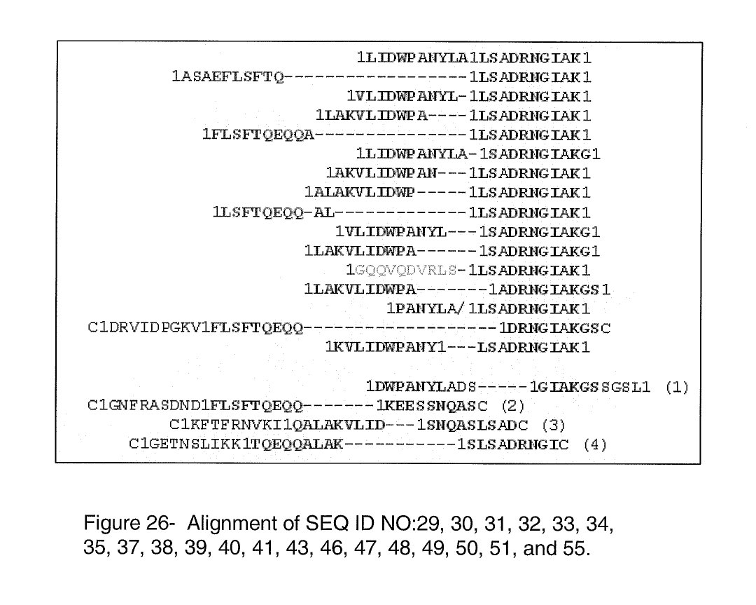

FIG. 24 illustrates 4 peaks illustrating specific peptides which were considered to be significant in that they gave good binding to the OPN-301 antibody (the amino acid sequences of these peaks are shown in FIG. 26 as SEQ ID NO: 49 to SEQ ID NO:52 respectively). From this analysis, a group of 20 CLIPS-peptides which were considered to bind to OPN-301 with the highest affinity were identified. These are shown in FIG. 25, these proteins being accorded SEQ ID NO:29 to SEQ ID NO:48, this numbering being such that SEQ ID NO:29 relates to the first listed peptide sequence, and SEQ ID NO:48 listed as the last listed peptide sequence at the foot of the table. The specific peptide is identified in the left hand column, while the binding affinity is shown in the right hand column (Rampo 1/1000, to OPN-301 monoclonal antibody present at 2 ug/ml).

Having identified the most specific binding peptides, an alignment of the amino acid sequences of these peptides is performed in order to identify areas of homology.

The result of this analysis is shown by the alignment in FIG. 26. In FIG. 25 and FIG. 26, a “C” and a “1” in the listed peptides both denote a cysteine residue. This analysis allows a determination of which regions are part of the proteins which are shown to be bound with the highest affinity by the TLR2 antibody. The identification of regions of amino acid sequences which are shown to be involved in binding allows these regions to be mapped to areas of the Toll-like Receptor 2 sequence. This shows that the amino acids which are bound by the OPN-301 antibody are present at the N-terminus and the C-terminus of the Toll-like Receptor 2 sequence. No peptides defining the LRR-regions appeared to be clearly recognised by the tested antibody. Furthermore, the identified binding regions were shown to contain various cysteine residues. Furthermore, the amino acid regions which are shown in this example of having a relatively high binding affinity to TLR2 were compared to the peptide sequences identified from Example 4 as having a high binding affinity. This analysis shows that the linear peptide which was identified in Example 4 as having the highest binding affinity to OPN-301 (the peptide having the amino acid sequence EFLSFTQEQQALAKV (SEQ ID NO: 12)) has areas of homology to the high affinity binding proteins defined herein. Furthermore, a further linear peptide identified as being a high affinity binding peptide in Example 4 shows homology with the peptides identified in the present examples as being a binding peptide, this peptide has the amino acid sequence EQQALAKVLIDWPAN (SEQ ID NO:13). It appears therefore that the results of Example 4, although suggesting that the epitope was not linear, did provide information to identify at least part of the epitope as the peptides of SEQ ID NO:12 and SEQ ID NO:13 concur with the peptides identified in the CLIPS analysis performed in the present example as defining the epitope to which OPN-301 has binding specificity. The other peptides, which were designated in Example 4 as SEQ ID NO:14 to SEQ ID NO:28 are likely to be linear peptides which mimic the binding regions identified in this example.

The results of this experiment therefore identify sequences of amino acids which are typically seen as being present in the peptides which are shown to bind with the greatest binding homology. In particular, 3 different regions of amino acids are often shown as being part of the peptides which bind with highest affinity, these regions are KEESSNQASLSCDRNGICKGS (SEQ ID NO:2), CSCEFLSFTQEQQ (SEQ ID NO:3) and ALAKVLIDWPANYL (SEQ ID NO:4).

It is therefore apparent that the epitope which is bound by antagonistic anti-TLR2 antibodies comprises sequences which are derived from at least one of SEQ ID NO:2, SEQ ID NO:3 or SEQ ID NO:4.

### Example 6

### Topology of Identified Binding Epitope

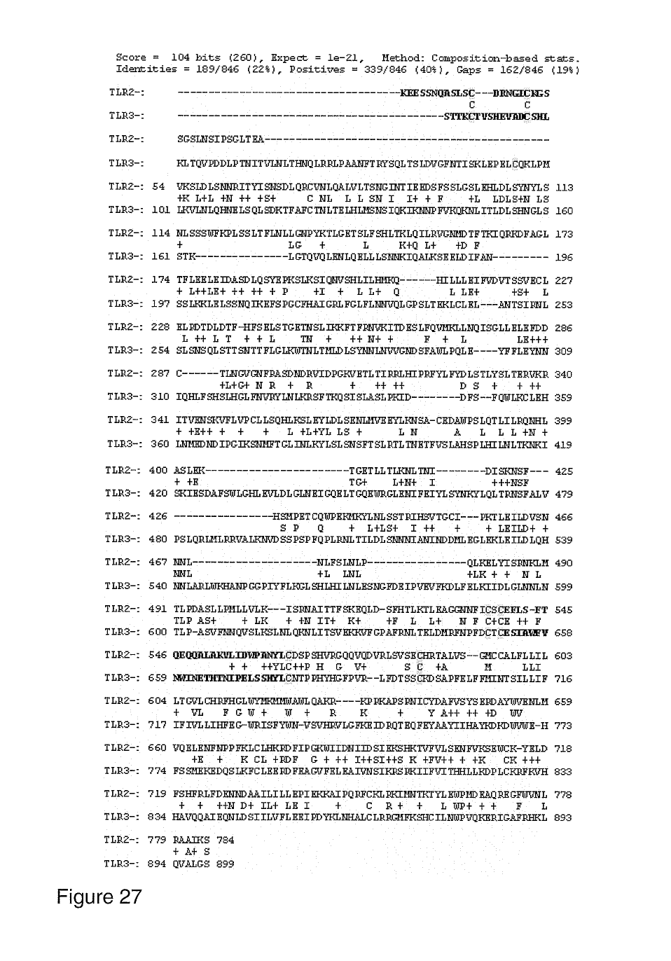

The three dimensional structure of human Toll-like Receptor 2 has not been definitively defined in the art to date. However, the structure of human Toll-like Receptor 3 is known in the art. FIG. 27 shows an amino acid homology alignment (using the BLAST software package which is well known to the person skilled in the art) to compare the homology of the defined human TLR2 and human TLR3 sequences.

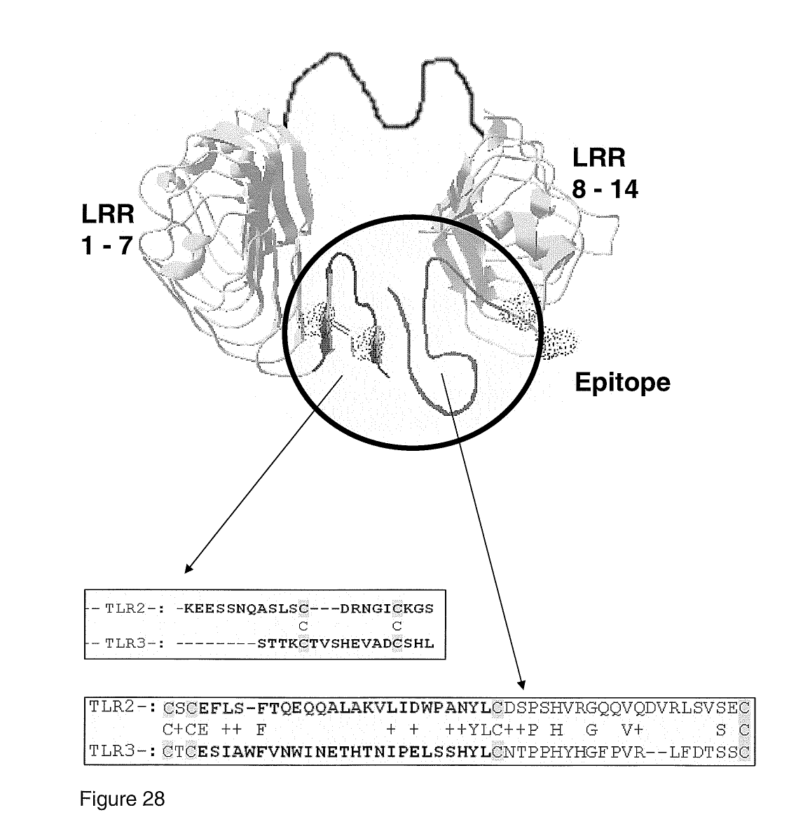

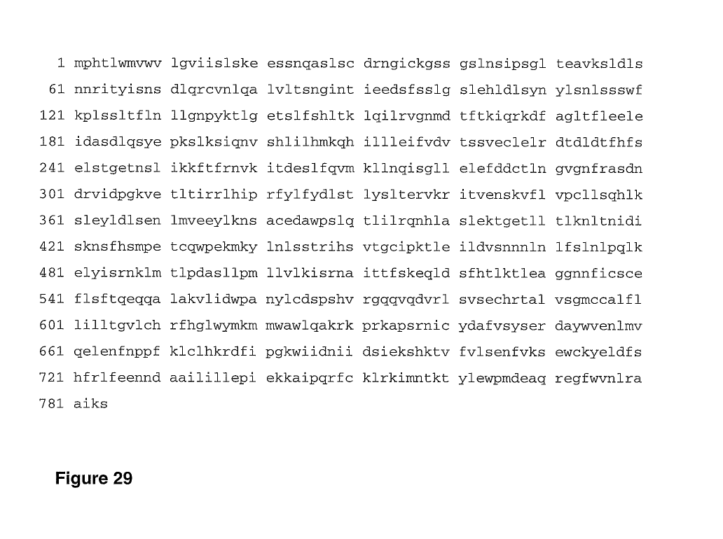

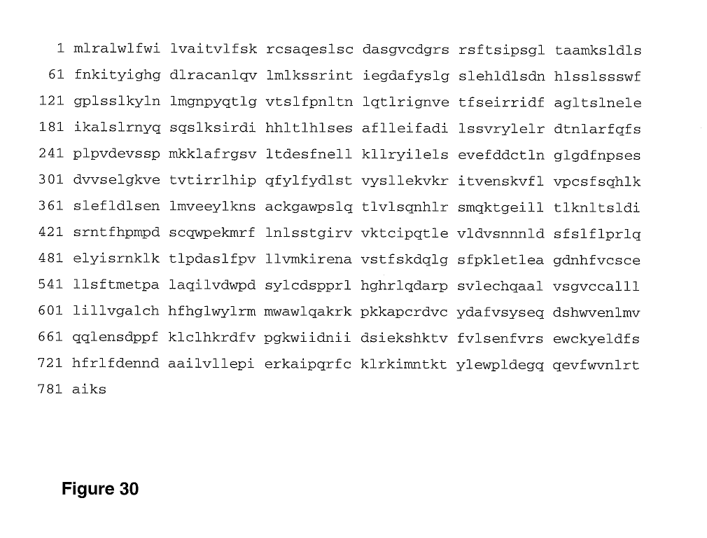

This alignment allows the structure of TLR2 to be predicted based on the elucidated structure of TLR3 which was previously published (Choe et al. Science. 2005. July 22, 309 (5754) pages 581-585 “Crystal Structure of Human Toll-Like Receptor 3 (TLR3) Ectodomain”). This comparison of sequences suggested that TLR2 has a different, but related structure to TLR3. Human TLR3 has 24 leucine rich repeat domains, while human TLR2 has only 14. FIG. 1(b) shows the predicted three dimensional structure of human TLR2. Human TLR2 only has 14 leucine rich repeats, however, these are grouped into 2, with a connecting region in between the groups, as represented in FIG. 1(a). FIG. 2 shows the probable location of disulphide bridges between cysteine residues. Furthermore, FIG. 28 shows a model of the predicted conformation of the epitope present in TLR2 which is bound by a TLR2 antagonistic antibody, the epitope comprising portions of both the C-terminus and N-terminus of the TLR2 polypeptide.

Based on the amino acid residues which are defined as being part of the epitope and the likely location of these residues within the TLR2 polypeptide, it is predicted that the epitope will be a conformational and discontinuous epitope.

All documents referred to in this specification are herein incorporated by reference. Modifications and variations to the described embodiments of the inventions will be apparent to those skilled in the art, without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A fragment of the Toll-like Receptor 2 (TLR2) receptor which consists of SEQ ID NO:2 and SEQ ID NO:5.

2-3. (canceled)

4. A binding member which specifically binds to an epitope of Toll-like Receptor 2 consisting of the amino acid sequence of SEQ ID NO:1 or SEQ ID NO:2 and one of SEQ ID NO:3, SEQ ID NO:4 or SEQ ID NO:5 and wherein the binding member is an antibody or an antibody binding fragment.

5. (canceled)

6. A polypeptide which defines an epitope of Toll-like Receptor 2 and which consists of the amino acid sequences SEQ ID NO:2 and SEQ ID NO:5.

7. A monoclonal antibody which has binding specificity for an epitope of Toll-like Receptor 2, said epitope consisting of the amino acid sequences SEQ ID NO:2 and SEQ ID NO:5.

8-10. (canceled)

11. The use of a polypeptide as claimed in claim 3 in a method for the producing a binding member which specifically binds to said polypeptide.

12. A method for the treatment or prophylaxis of a disease which is mediated by Toll-like Receptor 2 activation and/or signalling, the method comprising the step of administering to a subject in need of treatment a therapeutically effective amount of a binding member which specifically binds to an epitope consisting of SEQ ID NO:2 and SEQ ID NO:5.

13. (canceled)

14. A binding epitope which, when bound by binding compound having binding specificity for said epitope, results in antagonism of Toll-like Receptor 2 signalling function, said binding epitope consisting of an amino acid sequence of SEQ ID NO:2 and SEQ ID NO:5.

15-16. (canceled)

17. A binding member for use in the inhibition of the functional activity of Toll-like Receptor 2, said binding member having binding specificity for an epitope present on Toll-like Receptor 2 which comprises at least one amino acid sequence selected from the group consisting of SEQ ID NO:2 and SEQ ID NO:5, wherein binding of said binding member to Toll-like Receptor 2 results in an inhibition of the function of Toll-like Receptor 2 and wherein the binding member has binding specificity for Toll-like Receptor 2 irrespective of whether Toll-like Receptor 2 forms a heterodimer with Toll-like Receptor 1 or Toll-like Receptor 6.

18-23. (canceled)

24. A composition comprising a binding compound which has binding specificity for an epitope present on Toll-like Receptor 2 which comprises the amino acid sequence consisting of SEQ ID NO:2 and SEQ ID NO:5 for use in the treatment of a condition which is mediated by Toll-like Receptor 2 activation and signalling.

25. A pharmaceutical composition comprising a binding compound which inhibits Toll-like Receptor 2 function by binding to an epitope present on Toll-like Receptor 2 which consists of SEQ ID NO:2 and SEQ ID NO:5 along with at least one pharmaceutically acceptable carrier, diluent or excipient.

26. A pharmaceutical composition as claimed in claim 25 wherein the binding compound is selected from the group comprising: proteins, peptides, peptidomimetics, nucleic acids, polynucleotides, polysaccharides, oligopeptides, carbohydrates, lipids, small molecule compounds, and naturally occurring compounds.

27-28. (canceled)

29. An assay for assessing binding activity between an epitope present on Toll-like Receptor 2 which comprises the amino acid sequence of the amino acid of SEQ ID NO:1, 2, 4 or 5 and a putative binding molecule which comprises the steps of:
bringing at least one candidate binding compound into contact with the epitope which consists of the amino acid sequences of SEQ ID NO:2 and SEQ ID NO:5, and
determining interaction or binding between the at least one candidate binding compound and the epitope which consists of SEQ ID NO:2 and SEQ ID NO:5,
wherein binding between the at least one candidate binding compound and the epitope which consists of the amino acid sequences of SEQ ID NO:2 and SEQ ID NO:5 is indicative of the utility of the at least one candidate binding compound.

30. (canceled)

31. A vaccine composition comprising a polypeptide which comprises at least one of the amino acid sequences selected from the group consisting of SEQ ID NO:2 and SEQ ID NO:5.

32. A binding member as claimed in claim 4 which specifically binds to an epitope of Toll-like Receptor 2 consisting of the amino acid sequences of SEQ ID NO:2 and SEQ ID NO:5.

33. A binding member as claimed in claim 4 wherein the antibody or antibody binding fragment is a Toll-like Receptor 2 antagonist.

34. A binding member as claimed in claim 4 wherein the antibody or an antibody binding fragment binds to both the C terminal domain and N terminal domain of Toll-like Receptor 2 to an epitope consisting of the amino acid sequences of SEQ ID NO:2 and SEQ ID NO:5

35. A polypeptide as claimed in claim 6 wherein the Toll-like Receptor 2 epitope is a non-continuous epitope.

36. A method as claimed in claim 12 wherein the disease which is mediated by Toll-like Receptor 2 activation is sepsis.


SEQUENCE LISTING (55 sequences)

SEQ ID NO: 1 - protein, 5 aa, Homo sapiens
SLSCD

SEQ ID NO: 2 - protein, 21 aa, Homo sapiens
KEESSNQASLSCDRNGICKGS

SEQ ID NO: 3 - protein, 13 aa, Homo sapiens
CSCEFLSFTQEQQ

SEQ ID NO: 4 - protein, 14 aa, Homo sapiens
ALAKVLIDWPANYL

SEQ ID NO: 5 - protein, 27 aa, Homo sapiens
CSCEFLSFTQEQQALAKVLIDWPANYL

SEQ ID NO: 6 - DNA, 32 bp, Artificial Sequence
  Synthetic primer
  misc_feature: Cys30Ser (Substitution of cysteine residue at position 30 of human TLR2 to serine residue) primer
aggcttctctgtcttctgaccgcaatggtatc

SEQ ID NO: 7 - DNA, 33 bp, Artificial Sequence
  Synthetic primer
  misc_feature: Cys30Ser (Substitution of cysteine residue at position 30 of human TLR2 to serine residue) primer
gataccattgcggtcagaagacagagaagcctg

SEQ ID NO: 8 - DNA, 30 bp, Artificial Sequence
  Synthetic primer
  misc_feature: Cys36Ser (Substitution of cysteine residue at position 36 of human TLR2 to serine residue) primer
gaccgcaatggtatctgtaagggcagctca

SEQ ID NO: 9 - DNA, 30 bp, Artificial Sequence
  Synthetic primer
  misc_feature: Cys36Ser (Substitution of cysteine residue at position 36 of human TLR2 to serine residue) primer
tgagctgcccttacagataccattgcggtc

SEQ ID NO: 10 - DNA, 33 bp, Artificial Sequence
  Cys539Ser (Substitution of cysteine residue at position 539 of human TLR2 to serine residue) primer
aacttcatttgctcctctgaattcctctccttc

SEQ ID NO: 11 - DNA, 33 bp, Artificial Sequence
  Synthetic primer
  misc_feature: Cys539Ser (Substitution of cysteine residue at position 539 of human TLR2 to serine residue) primer
gaaggagaggaattcagaggagcaaatgaagtt

SEQ ID NO: 12 - protein, 15 aa, Artificial Sequence
  Synthetic peptide
  MISC_FEATURE: Artificial peptide sequence which has binding affinity to human Toll-like Receptor 2
EFLSFTQEQQALAKV

SEQ ID NO: 13 - protein, 15 aa, Artificial Sequence
  Synthetic peptide
  MISC_FEATURE: Artificial peptide sequence which has binding affinity to human Toll-like Receptor 2
EQQALAKVLIDWPAN

SEQ ID NO: 14 - protein, 15 aa, Artificial Sequence
  Synthetic peptide
  MISC_FEATURE: Artificial peptide sequence which has binding affinity to human Toll-like Receptor 2
FLSFTQEQQALAKVL

SEQ ID NO: 15 - protein, 15 aa, Artificial Sequence
  Synthetic peptide
  MISC_FEATURE: Artificial peptide sequence which has binding affinity to human Toll-like Receptor 2
TFSKEQLDSFHTLKT

SEQ ID NO: 16 - protein, 15 aa, Artificial Sequence
  Synthetic peptide
TTFSKEQLDSFHTLK

SEQ ID NO: 17 - protein, 15 aa, Artificial Sequence
  Synthetic peptide
  MISC_FEATURE: Artificial peptide sequence which has binding affinity to human Toll-like Receptor 2
YLCDSPSHVRGQQVQ

SEQ ID NO: 18 - protein, 15 aa, Artificial Sequence
  Synthetic peptide
  MISC_FEATURE: Artificial peptide sequence which has binding affinity to human Toll-like Receptor 2
NYLCDSPSHVRGQQV

SEQ ID NO: 19 - protein, 15 aa, Artificial Sequence
  Synthetic peptide
  MISC_FEATURE: Artificial peptide sequence which has binding affinity to human Toll-like Receptor 2
ANYLCDSPSHVRGQQ

SEQ ID NO: 20 - protein, 15 aa, Artificial Sequence
  Synthetic peptide
  MISC_FEATURE: Artificial peptide sequence which has binding affinity to human Toll-like Receptor 2
ASDLQSYEPKSLKSI

SEQ ID NO: 21 - protein, 15 aa, Artificial Sequence
  Synthetic peptide
  MISC_FEATURE: Artificial peptide sequence which has binding affinity to human Toll-like Receptor 2
SDLQSYEPKSLKSIQ

SEQ ID NO: 22 - protein, 15 aa, Artificial Sequence
  Synthetic peptide
  MISC_FEATURE: Artificial peptide sequence which has binding affinity to human Toll-like Receptor 2
IDASDLQSYEPKSLK

SEQ ID NO: 23 - protein, 15 aa, Artificial Sequence
  Synthetic peptide
  MISC_FEATURE: Artificial peptide sequence which has binding affinity to human Toll-like Receptor 2
DASDLQSYEPKSLKS

SEQ ID NO: 24 - protein, 15 aa, Artificial Sequence
  Synthetic peptide
  MISC_FEATURE: Artificial peptide sequence which has binding affinity to human Toll-like Receptor 2
LQSYEPKSLKSIQNV

SEQ ID NO: 25 - protein, 15 aa, Artificial Sequence
  Synthetic peptide
  MISC_FEATURE: Artificial peptide sequence which has binding affinity to human Toll-like Receptor 2
HSMPETCQWPEKMKY

SEQ ID NO: 26 - protein, 15 aa, Artificial Sequence
  Synthetic peptide
  MISC_FEATURE: Artificial peptide sequence which has binding affinity to human Toll-like Receptor 2
PETCQWPEKMKYLNL

SEQ ID NO: 27 - protein, 15 aa, Artificial Sequence
  Synthetic peptide
  MISC_FEATURE: Artificial peptide sequence which has binding affinity to human Toll-like Receptor 2
SMPETCQWPEKMKYL

SEQ ID NO: 28 - protein, 15 aa, Artificial Sequence
  Synthetic peptide
  MISC_FEATURE: Artificial peptide sequence which has binding affinity to human Toll-like Receptor 2
MPETCQWPEKMKYLN

SEQ ID NO: 29 - protein, 23 aa, Artificial Sequence
  Synthetic peptide
  MISC_FEATURE: Artificial peptide sequence which has binding affinity to human Toll-like Receptor 2
CLIDWPANYLACLSADRNGIAKC

SEQ ID NO: 30 - protein, 23 aa, Artificial Sequence
  Synthetic peptide
  MISC_FEATURE: Artificial peptide sequence which has binding affinity to human Toll-like Receptor 2
CASAEFLSFTQCLSADRNGIAKC

SEQ ID NO: 31 - protein, 23 aa, Artificial Sequence
  Synthetic peptide
  MISC_FEATURE: Artificial peptide sequence which has binding affinity to human Toll-like Receptor 2
CVLIDWPANYLCLSADRNGIAKC

SEQ ID NO: 32 - protein, 23 aa, Artificial Sequence
  Synthetic peptide
  MISC_FEATURE: Artificial peptide sequence which has binding affinity to human Toll-like Receptor 2
CLAKVLIDWPACLSADRNGIAKC

SEQ ID NO: 33 - protein, 18 aa, Artificial Sequence
  Synthetic peptide
  MISC_FEATURE: Artificial peptide sequence which has binding affinity to human Toll-like Receptor 2
CFLSFTQEQQACLSADRN

SEQ ID NO: 34 - protein, 24 aa, Artificial Sequence
  Synthetic peptide
  MISC_FEATURE: Artificial peptide sequence which has binding affinity to human Toll-like Receptor 2
CLIDWPANYLACSADRRNGIAKGC

SEQ ID NO: 35 - protein, 23 aa, Artificial Sequence
  Synthetic peptide
  MISC_FEATURE: Artificial peptide sequence which has binding affinity to human Toll-like Receptor 2
CAKVLIDWPANCLSADRNGIAKC

SEQ ID NO: 36 - protein, 17 aa, Artificial Sequence
  Synthetic peptide
  MISC_FEATURE: Artificial peptide sequence which has binding affinity to human Toll-like Receptor 2
CSKVFLVPALLSQHLKC

SEQ ID NO: 37 - protein, 23 aa, Artificial Sequence
  Synthetic peptide
  MISC_FEATURE: Artificial peptide sequence which has binding affinity to human Toll-like Receptor 2
CALAKVLIDWPCLSADRNGIAKC

SEQ ID NO: 38 - protein, 23 aa, Artificial Sequence
  Synthetic peptide
  MISC_FEATURE: Artificial peptide sequence which has binding affinity to human Toll-like Receptor 2
CLSFTQEQQALCLSADRNGIAKC

SEQ ID NO: 39 - protein, 23 aa, Artificial Sequence
  Synthetic peptide
  MISC_FEATURE: Artificial peptide sequence which has binding affinity to human Toll-like Receptor 2
CVLIDWPANYLCSADRNGIAKGC

SEQ ID NO: 40 - protein, 23 aa, Artificial Sequence
  Synthetic peptide
  MISC_FEATURE: Artificial peptide sequence which has binding affinity to human Toll-like Receptor 2
CLAKVLIDWPACSADRNGIAKGC

SEQ ID NO: 41 - protein, 23 aa, Artificial Sequence
  Synthetic peptide
  MISC_FEATURE: Artificial peptide sequence which has binding affinity to human Toll-like Receptor 2
CGQQVQDVRLSCLSADRNGIAKC

SEQ ID NO: 42 - protein, 33 aa, Artificial Sequence
  Synthetic peptide
  MISC_FEATURE: Artificial peptide sequence which has binding affinity to human Toll-like Receptor 2
CIQNVSHLILHMKQHCGCLKNLTNIDISKNSFC

SEQ ID NO: 43 - protein, 23 aa, Artificial Sequence
  Synthetic peptide
  MISC_FEATURE: Artificial peptide sequence which has binding affinity to human Toll-like Receptor 2
CLAKVLIDWPACADRNGIAKGSC

SEQ ID NO: 44 - protein, 33 aa, Artificial Sequence
  Synthetic peptide
  MISC_FEATURE: Artificial peptide sequence which has binding affinity to human Toll-like Receptor 2
CIQNVSHLILHMKQHCGCLPQLKELYISRNKLC

SEQ ID NO: 45 - protein, 17 aa, Artificial Sequence
  Synthetic peptide
  MISC_FEATURE: Artificial peptide sequence which has binding affinity to human Toll-like Receptor 2
CMVQELENFNPPFKLAC

SEQ ID NO: 46 - protein, 23 aa, Artificial Sequence
  Synthetic peptide
CPANYLADSPSCLSADRNGIAKC

SEQ ID NO: 47 - protein, 32 aa, Artificial Sequence
  Synthetic peptide
  MISC_FEATURE: Artificial peptide sequence which has binding affinity to human Toll-like Receptor 2
CCDRVIDPGKVCFLSFTQEQQCDRNGIAKGSC

SEQ ID NO: 48 - protein, 23 aa, Artificial Sequence
  Synthetic peptide
  MISC_FEATURE: Artificial peptide sequence which has binding affinity to human Toll-like Receptor 2
CKVLIDWPANYCLSADRNGIAKC

SEQ ID NO: 49 - protein, 23 aa, Artificial Sequence
  Synthetic peptide
  MISC_FEATURE: Artificial peptide sequence which has binding affinity to human Toll-like Receptor 2
CDWPANYLADSCGIAKGSSKSLC

SEQ ID NO: 50 - protein, 32 aa, Artificial Sequence
  Synthetic peptide
  MISC_FEATURE: Artificial peptide sequence which has binding affinity to human Toll-like Receptor 2
CCGNFRASDNDCFLSFTQEQQCKEESSNQASC

SEQ ID NO: 51 - protein, 32 aa, Artificial Sequence
  Synthetic peptide
  MISC_FEATURE: Artificial peptide sequence which has binding affinity to human Toll-like Receptor 2
CCFKTFRNVKICQALAKVLIDCSNQASLSADC

SEQ ID NO: 52 - skipped

SEQ ID NO: 53 - protein, 784 aa, Homo sapiens [sequence omitted]

SEQ ID NO: 54 - protein, 784 aa, Mus musculus
MLRALWLFWILVAITVLFSKRCSAQESLSCDASGVCDGRSRSFTSIPSGLTAAMKSLDLSFNKITYIGHGDLRACANLQVLMLKSSRINTIEGDAFYSLGSLEHLDLSDNHLSSLSSSWFGPLSSLKYLNLMGNPYQTLGVTSLFPNLTNLQTLRIGNVETFSEIRRIDFAGLTSLNELEIKALSLRNYQSQSLKSIRDIHHLTLHLSESAFLLEIFADILSSVRYLELRDTNLARFQFSPLPVDEVSSPMKKLAFRGSVLTDESFNELLKLLRYILELSEVEFDDCTLNGLGDFNPSESDVVSELGKVETVTIRRLHIPQFYLFYDLSTVYSLLEKVKRITVENSKVFLVPCSFSQHLKSLEFLDLSENLMVEEYLKNSACKGAWPSLQTLVLSQNHLRSMQKTGEILLTLKNLTSLDISRNTFHPMPDSCQWPEKMRFLNLSSTGIRVVKTCIPQTLEVLDVSNNNLDSFSLFLPRLQELYISRNKLKTLPDASLFPVLLVMKIRENAVSTFSKDQLGSFPKLETLEAGDNHFVCSCELLSFTMETPALAQILVDWPDSYLCDSPPRLHGHRLQDARPSVLECHQAALVSGVCCALLLLILLVGALCHHFHGLWYLRMMWAWLQAKRKPKKAPCRDVCYDAFVSYSEQDSHWVENLMVQQLENSDPPFKLCLHKRDFVPGKWIIDNIIDSIEKSHKTVFVLSENFVRSEWCKYELDFSHFRLFDENNDAAILVLLEPIERKAIPQRFCKLRKIMNTKTYLEWPLDEGQQEVFWVNLRTAIKS

SEQ ID NO: 55 - protein, 32 aa, Artificial Sequence
  Synthetic peptide
  MISC_FEATURE: Artificial peptide sequence which has binding affinity to human Toll-like Receptor 2
CCGETNSLIKKCTQEQQALAKCSLSADRNGIC